(19) United States
(12) Patent Application Publication
(10) Pub. No.: US 2025/0237667 A1
(43) Pub. Date: Jul. 24, 2025

(54) THERAPEUTIC AND DIAGNOSTIC METHODS AND COMPOSITIONS FOR NEURODEGENERATIVE DISEASES

(71) Applicant: WISCONSIN ALUMNI RESEARCH FOUNDATION, Madison, WI (US)

(72) Inventors: Su-Chun Zhang, Waunakee, WI (US); Hong CHen, Madison, WI (US)

(21) Appl. No.: 19/063,260

(22) Filed: Feb. 25, 2025

Related U.S. Application Data
(63) Continuation of application No. 17/313,587, filed on May 6, 2021, now Pat. No. 12,247,986.
Application No. 17/313,587 is a continuation of application No. 16/012,467, filed on Jun. 19, 2018, now Pat. No. 11,002,745.
Application No. 16/012,467 is a division of application No. 14/676,569, filed on Apr. 1, 2015, now Pat. No. 10,024,870.
(60) Provisional application No. 61/974,296, filed on Apr. 2, 2014.

(51) Int. Cl. G01N 33/68; A61K 38/17

(52) U.S. Cl. CPC G01N 33/6896; A61K 38/1709; G01N 2333/47; G01N 2800/28

(57) ABSTRACT

Methods and compositions relating to motor neurons derived from induced pluripotent stem cells of subjects having a neurodegenerative disease, where the motor neurons exhibit phenotypes characteristic of the neurodegenerative disease, are provided herein. In particular, the present invention provides methods for screening putative therapeutic agents and methods for diagnosing living subjects as having a neurodegenerative disease. In addition, the present invention provides therapeutic gene transfer methods for treating or preventing a neurodegenerative disease in a subject in need thereof.

### CROSS-REFERENCE TO RELATED APPLICATIONS

This application is a divisional application of U.S. patent application Ser. No. 14/676,569, filed Apr. 1, 2015, which claims the benefit of U.S. Provisional Patent Application No. 61/974,296, filed Apr. 2, 2014, each of which is incorporated herein by reference as if set forth in its entirety.

### STATEMENT REGARDING FEDERALLY SPONSORED RESEARCH OR DEVELOPMENT

Not applicable.

### BACKGROUND OF THE INVENTION

Neurodegenerative disease is a term that encompasses a range of pathologies that primarily affect neurons of the central nervous system. Neurodegenerative diseases are typically characterized by the progressive degeneration and/or death of neurons in different regions of the nervous system and the resulting impairments in movement and mental functioning. Neurodegenerative diseases, which are incurable and often debilitating, have an enormous impact on the lives of affected individuals and their families as well as society as a whole.

Parkinson's disease, amyotrophic lateral sclerosis (ALS), and Alzheimer's disease are the most well-known neurodegenerative diseases, but other conditions, such as Spinal Muscle Atrophy, Charcot-Marie-Tooth disease, Huntington's disease, spinocerebellar ataxias, Guillain-Barre syndrome, Parkinson's disease-related disorders, and other motor neuron diseases belong to the same clinical group. Pathologies common to genetically inherited and sporadic cases of these neurodegenerative diseases are the accumulation of misfolded proteins, especially neurofilament (NF), and axonal degeneration. It remains unknown how protein aggregation, mitochondrial dysfunction, glutamate toxicity, and disrupted calcium homeostasis promote axonal degeneration or why these processes selectively affect specific populations of neurons, such as motor neurons in ALS, although some evidence suggests that protein aggregates disrupt axonal transportation and consequently promote retraction of motor neuron axonal degeneration before the loss of cell bodies.

Accordingly, there remains a need for a better understanding of the etiopathology of neurodegenerative diseases. In addition, there remains a need in the art for methods for detecting neurodegenerative conditions before clinical symptoms manifest, for facilitating accurate diagnosis of neurodegenerative disease, for monitoring disease progression, and for identifying candidate therapeutic agents to slow, halt, or reverse neurodegeneration.

### SUMMARY OF THE INVENTION

In one aspect, the present invention provides a method of diagnosing neurodegenerative disease in a subject. The method can comprise the steps of obtaining induced pluripotent stem (iPS) cells from somatic cells of a subject, where the iPS cells are capable of differentiation into neurons; culturing the iPS cells under conditions suitable to differentiate the iPS cells into neurons; and detecting an indicator of neurofilament aggregation or neurite degeneration in the iPS cell-derived neurons, where increased neurofilament aggregation or neurite degeneration relative to neurons derived from iPS cells of an individual not having neurodegenerative disease indicates that the subject has a neurodegenerative disease, and thereby diagnosing neurodegenerative disease in the subject.

In some cases, the neurodegenerative disease can be selected from the group consisting of amyotrophic lateral sclerosis (ALS), Alzheimer's Disease (AD), Parkinson's Disease (PD), a PD-related disorder, Huntington's Disease (HD), Charcot-Marie-Tooth disease (CMT), Spinocerebellar ataxia (SCA), Spinal Muscle Atrophy (SMA), and Guillain-Barré syndrome (GBS). The neurodegenerative disease can be ALS, SMA, or CMT, and the subject's iPS cells can differentiate into motor neurons. Detecting neurofilament aggregation can comprise determining a level of NF-L mRNA in the subject's iPS cell-derived motor neurons. In some cases, the method can further comprise detecting a level of phosphorylated neurofilament in a biological sample of the subject. The biological sample of the subject can be cerebrospinal fluid. The neurodegenerative disease can be Alzheimer's disease, and the subject's iPS cells can differentiate into glutamatergic neurons or cholinergic neurons. The neurodegenerative disease can be Parkinson's disease or a PD-related disorder, and the subject's iPS cells can differentiate into dopaminergic neurons. The neurodegenerative disease can be spinocerebellar ataxia and the subject's iPS cells can differentiate into granular neurons.

In another aspect, the present invention provides a method of detecting a neurodegenerative disease in a subject. The method can comprise the steps of determining a level of NF-L mRNA in neurons derived from iPS cells obtained from somatic cells of a subject; relating the determined level to a reference level of NF-L; and thereby detecting neurodegenerative disease in the subject based on a reduced level of NF-L relative to the reference level. The neurodegenerative disease can be selected from the group consisting of amyotrophic lateral sclerosis (ALS), Alzheimer's Disease (AD), Parkinson's Disease (PD), a PD-related disorder, Huntington's Disease (HD), Charcot-Marie-Tooth disease (CMT), Spinocerebellar ataxia (SCA), Spinal Muscle Atrophy (SMA), and Guillain-Barré syndrome (GBS). In some cases, the neurodegenerative disease is ALS, where the neurons can be motor neurons, and where the reference level can be a level of NF-L mRNA in motor neurons derived from iPS cells of an individual having ALS. The reduced level of NF-L mRNA can be at least 50% lower than the reference.

In a further aspect, the present invention provides a method for treating a neurodegenerative disease in a subject in need thereof. The method can comprise administering one or more recombinant nucleic acid sequences encoding at least a portion of NF-L to the subject, where the nucleic acid sequences can be targeted to neurons, and where expression of NF-L in targeted neurons can treat the neurodegenerative disease. The neurodegenerative disease can be selected from the group consisting of amyotrophic lateral sclerosis (ALS), Alzheimer's Disease (AD), Parkinson's Disease (PD), a PD-related disorder, Huntington's Disease (HD), Charcot-Marie-Tooth disease (CMT), Spinocerebellar ataxia (SCA), Spinal Muscle Atrophy (SMA), and Guillain-Barre syndrome (GBS). The nucleic acid sequences can be administered in a vector. The vector can be a virus or virus-derived. The virus can be selected from the group consisting of an adenovirus, retrovirus, herpes virus, and adeno-associated virus. The vector can be a replication defective adenovirus.

In another aspect, the present invention provides a method for protecting against neurite degeneration in a subject in need thereof. The method can comprise administering one or more nucleic acid sequences encoding NF-L to tissue of the subject, where the nucleic acid sequences are targeted to neurons of the tissue, and where expression of NF-L protects the targeted neurons from neurite degeneration. The subject can have been diagnosed or can be suspected of having a neurodegenerative disease. The neurodegenerative disease can be selected from the group consisting of amyotrophic lateral sclerosis (ALS), Alzheimer's Disease (AD), Parkinson's Disease (PD), a PD-related disorder, Huntington's Disease (HD), Charcot-Marie-Tooth disease (CMT), Spinocerebellar ataxia (SCA), Spinal Muscle Atrophy (SMA), and Guillain-Barre syndrome (GBS). The nucleic acid sequences can be administered in a vector. The vector is a virus or virus-derived. The virus can be selected from the group consisting of an adenovirus, retrovirus, herpes virus, and adeno-associated virus.

In a further aspect, the present invention provides a method of evaluating a candidate neuroprotective agent. The method can comprise the steps of contacting a candidate neuroprotective agent to neurons derived from induced pluripotent stem (iPS) cells obtained from somatic cells of a human subject having a neurodegenerative disease, where the neurons exhibit a phenotype typical of the neurodegenerative disease; and evaluating the contacted neurons for a neuroprotective effect of the agent relative to non-contacted iPS cell-derived neurons of the subject. The neurodegenerative disease can be selected from the group consisting of amyotrophic lateral sclerosis (ALS), Alzheimer's Disease (AD), Parkinson's Disease (PD), a PD-related disorder, Huntington's Disease (HD), Charcot-Marie-Tooth disease (CMT), Spinocerebellar ataxia (SCA), Spinal Muscle Atrophy (SMA), and Guillain-Barré syndrome (GBS). The neuroprotective effect can be selected from the group consisting of a reduction in severity of neurodegeneration, a delay in onset of neurodegeneration, a reduction in severity of neurofilament (NF) aggregation, and increased motor neuron viability in vitro.

In another aspect, the present invention provides a recombinant nucleic acid molecule comprising a motor neuron-specific promoter operably linked to a nucleic acid sequence encoding a human NF-L polypeptide. The invention also provides a vector comprising said nucleic acid molecule. The vector can be a plasmid. The vector can be a virus or virus-derived. The virus can be selected from the group consisting of an adenovirus, retrovirus, herpes virus, and adeno-associated virus. The vector can be a replication defective adenovirus.

In a further aspect, the present invention provides a kit for diagnosing a subject predisposed to or suspected of developing a neurodegenerative disease or suffering from a neurodegenerative disease. The kit can comprise at least one oligonucleotide primer capable of hybridizing to a at least a portion of a NF-L target nucleic acid; at least one reference corresponding to a level of NF-L target nucleic acid; at least one buffer or reagent; and a container. The at least one oligonucleotide primer can comprise the nucleotide sequence of any of SEQ ID NO:7-8. The neurodegenerative disease can be selected from the group consisting of ALS, SMA, and CMT.

### BRIEF DESCRIPTION OF THE DRAWINGS

FIGS. 1A-1I demonstrate iPS cell generation, neural differentiation, and mutation correction. (FIG. 1A) Contrast image of iPS cell colonies generated by Sendai virus. (FIG. 1B) Immunofluorescent image of NANOG expression in D90A SOD1 iPS cells. (FIGS. 1C-D) DNA sequencing showing heterozygous nucleotides (A/C) in D90A (C) and homozygous nucleotide A in corrected (D90D) (D) SOD1 iPS cells. (FIG. 1E) Schematic protocol for MN and non-MN differentiation. 3c: 3 small molecular compounds (SB431542, LDN193189, and CHIR99021); Pur: purmorphamine; Cyclo: cyclopamine. (FIGS. 1F-G) ALS (D90A) and genetically corrected ALS (D90D) iPS cells differentiated to OLIG2⁺ MN progenitors at day−14, MNX1⁺ postmitotic MNs at day−21, and CHAT⁺ maturing MNs at day−28. (FIGS. 1H-I) Quantification of TUJ⁺ neuronal population among total Hoechst labeled (HO) cells (FIG. 1H) and MNX1⁺ MNs among neurons (FIG. 1I). Scale bar=50 μm.

FIGS. 2A-2D demonstrate SOD1 expression and aggregation in iPSC-derived neurons. (FIG. 2A) Allelic imbalance assay showing the ratio of mutant (A) and wt (C) copy of SOD1 transcripts in fibroblasts (FIB), reprogrammed stem cells (iPS), and their differentiated neuroepithelia (NEP), MN progenitors (MNP), MNs, and non-MNs. (FIG. 2B) RT-qPCR analysis showing SOD1 mRNA expression in MNs and non-MNs. (FIG. 2C) Representative Western blots and relative SOD1 expression levels (to GAPDH) in MNs and non-MNs. (FIG. 2D) SOD1 immuno-EM in neurites, cytoplasm, nuclei, and mitochondria of MN and non-MN cultures. Arrows=clusters of gold particles. No contrast staining for ALS non-MNs to permit better views of fine gold particles. Scale bar=2 μm.

FIGS. 3A-3D depict neurofilament (NF) aggregates in ALS iPSC-derived neurons. (FIG. 3A) Immunofluorescent images of NF-H, NF-M, and NF-L in CHAT⁺ MNs. NF staining in the insets is magnified on the right panel. Arrows indicate NF aggregates in the cell body; arrowheads indicate NF aggregates in neurites. Scale bar=50 μm. (FIG. 3B) EM showing NF arrangement in cell body (left) and neurites (right) of MN cultures. Scale bar=2 μm. (FIGS. 3C-D) Quantification of NF aggregate-containing cell bodies (FIG. 3C) and neurites (FIG. 3D) in MNs and non-MNs at day−4, 7, and 10 after plating. *p<0.05.

FIGS. 4A-4G depict degenerative changes in ALS MN neurites. (FIG. 4A) Colorimetric measurement of LDH (normalized to gDNA) in culture media from MN and non-MN cultures. (FIG. 4B) Cleaved caspase3 staining (arrows) and (FIG. 4C) quantification. (FIG. 4D) Phase contrast images of MNs and non-MNs at day−10. Arrows indicate bead-like swellings in neurites. Inset is magnified in upper-right. (FIG. 4E) Immunofluorescent images of p-NF-H in MNs and non-MNs. Arrowheads indicate bead-like structures in neurites. Inset magnified in upper right. (FIG. 4F) Quantification of beads on neurites. (FIG. 4G) ELISA quantification of p-NF-H in media (normalized to gDNA). *p<0.01. Scale bar=50 μm.

FIGS. 5A-5G present images demonstrating NF aggregation and neurite degeneration in neurons expressing D90A SOD1 in a wild-type (wt) background. (FIG. 5A) Western blots and relative expression of SOD1 (to GPDH) in MNs and non-MNs derived from hESCs expressing D90A SOD1 or EGFP. (FIG. 5B) Immunofluorescent images of NF-H, NF-M, and NF-L in CHAT⁺ cells from SOD1- and EGFP-expressing hESCs. NF staining in the insets is magnified on the right panel. Arrows indicate NF aggregates in the cell body; arrowheads indicate NF aggregates in neurites. (FIGS. 5C, D) Quantification of NF aggregate-containing cell bodies (FIG. 5C) and neurites (FIG. 5D) at day−4, 7, and 10 after plating neurons. (FIG. 5E) Phase contrast images of MNs and non-MNs from mutant SOD1- and EGFP-expressing hESCs. Arrows indicate bead-like formations in neurites. Inset is magnified in upper-right. (FIGS. 5F, G) p-NF-H (FIG. 5F) and LDH (FIG. 5G) in culture media from MN and non-MN cultures derived from SOD1- or EGFP-expressing hESCs. *p<0.05; **p<0.01. Scale bar=50 μm.

FIGS. 6A-6F present data demonstrating expression of NF subunits in neurons. (FIG. 6A) Relative levels of mRNAs for NF-L, NF-H, and NF-M, and β-actin mRNA in ALS (D90A) and corrected (D90D) MNs in the presence of actinomycin D measured by RT-qPCR (p<0.05 between D90A and D90D). (FIG. 6B) Relative expression and representative Western blots of NF-H, NF-M, and NF-L in MNs and non-MNs as compared to neuron-specific enolase (NSE). (FIG. 6C) The proportion of NF-L among total NF protein in MNs (upper panel) and non-MNs (lower panel). *p<0.05 between ALS (D90A, A4V) and genetically corrected ALS (D90D) or wt (IMR-90-4) cells. (FIG. 6D) Relative levels of NF-L and β-actin mRNA in ALS (D90A) and corrected (D90D) MNs in the presence of actinomycin D measured by RT-qPCR (p<0.05 between D90A and D90D). (FIG. 6E) Western blotting for mutant SOD1 in MN and non-MN samples pulled down by the 3′UTR NF-L mRNA probe. (FIG. 6F) Input samples were blotted for A5C3 antibody.

FIGS. 7A-7H present data demonstrating the effects of NF-L expression on NF aggregation and neurite degeneration in ALS MNs. (FIG. 7A) Western blots and relative (to neuron-specific enolase (NSE)) expression of NF-H, NF-M, and NF-L in MNs from GFP-, NF-L-expressing ALS (D90A) iPS cells as well as genetically corrected (D90D1 & 2) ALS iPS cells. (FIG. 7B) The proportion of NF-L among total NF protein in the presence of 1 μg/ml of DOX. (C, D) Quantification of NF aggregate-containing cell bodies (FIG. 7C) and neurites (FIG. 7D) in MNs. (FIG. 7E) Phase contrast images of MNs at day-10. Arrows indicate bead-like swellings in neurites. Inset is magnified in upper-right. (FIG. 7F) Immunofluorescent images of pNF-H in MNs. Arrowheads indicate bead-like structures in neurites. (FIG. 7G) Quantification of bead-like formations. (FIG. 7H) ELISA quantification of pNF-H in media from MN cultures. Scale bar=50 μm. *p<0.05.

FIGS. 8A-8C present data to demonstrate that sporadic ALS motor neurons exhibit NF aggregates and changes in NF-L mRNA levels. (FIG. 8A) Immunofluorescent staining for NF-L in sporadic and control (IMR90) MNs at day−10. Inset is magnified on the right. Arrow indicates aggregate in cell body and arrowhead indicates inclusion in neurite. (FIG. 8B) Electron micrograph shows NF aggregate in a MN neurite from ALS but not control (IMR-90). (FIG. 8C) Quantification of NF-L mRNA by RT-qPCR between control (IMR-90) and sporadic ALS MNs.

FIGS. 9A-9B present (FIG. 9A) nucleotide (SEQ ID NO:1) and (FIG. 9B) amino acid (SEQ ID NO:2) sequences for NF-L.

FIGS. 10A-10B present (FIG. 10A) nucleotide (SEQ ID NO:3) and (FIG. 10B) amino acid (SEQ ID NO:4) sequence for NF-H.

FIGS. 11A-11B present (FIG. 11A) nucleotide (SEQ ID NO:5) and (FIG. 11B) amino acid (SEQ ID NO:6) sequences for NF-M.

FIGS. 12A-12F present immunofluorescent images of NF-H, NF-M, and NF-L in CHAT⁺ motor neurons (MNs) and CHAT-non-MN from wild-type (FIGS. 12A, D), TDP43 mutant (FIGS. 12B, E), and sporadic ALS subjects (FIGS. 12C, F). Staining in the insets is magnified on the right panel. Arrows indicate NF inclusions in the cell body; arrowheads indicate NF inclusions in neurites. Scale bar=50 μm.

### DETAILED DESCRIPTION OF THE INVENTION

The present invention is based, at least in part, on the Inventors' discovery that motor neurons differentiated from induced pluripotent stem cells of ALS patients exhibit phenotypes characteristic of neurodegenerative diseases such as MN-selective accumulation of neurofilament (NF) protein and axonal degeneration. The Inventors further discovered that ALS-iPS cells exhibit altered proportions of NF subunits and significantly reduced levels of NF-low (NF-L). Using induced pluripotent stem (iPS) cells derived from ALS patients, including those with genetic mutations (SOD1 and TDP43) and without genetic mutations (sporadic), the Inventors discovered a motor neuron-selective NF aggregation at early stages and showed that these motor neurons gradually undergo axonal degeneration. They further discovered that ALS motor neurons show reduced expression of neurofilament-low (NF-L) and altered proportion of NF subunits (low, medium and high molecular weight). Importantly, they found that correction of NF-L mRNA expression in motor neurons restores the regular proportion of NF subunits, prevents NF aggregation, and subsequently protects motor neurons from undergoing degeneration.

To date, animal models have failed to fully recapitulate the neuropathology observed in human patients having neurodegenerative diseases. As described herein, ALS-iPS cells retain the ALS disease phenotype and have the capacity for differentiation into neuronal cells that exhibit a phenotype typical of ALS. For example, motor neurons derived from ALS-iPS cells undergo continual degeneration over time. The Examples below disclose that iPS cells generated from a human patient with ALS can be differentiated into motor neurons that then exhibit disease-specific phenotypes and undergo disease-specific cell death in the culture dish. Accordingly, the work described herein represents a very powerful example of a model in which to explore neurodegenerative disease mechanisms and to screen for novel compounds that may attenuate or block neurodegenerative disease processes.

In the specification and in the claims, the terms “including” and “comprising” are open-ended terms and should be interpreted to mean “including, but not limited to . . . ” These terms encompass the more restrictive terms “consisting essentially of” and “consisting of.”

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art.

### Methods of the Invention

a. Treatment Methods

One aspect of the present invention relates to methods for treating or preventing a neurodegenerative disease and for protecting against axonal degeneration in a subject in need thereof. As used herein, the terms “treat” and “treating” refer to both therapeutic treatment and prophylactic or preventative measures, wherein the object is to prevent or slow down (lessen) an undesired physiological change or disorder, such as progressive neurodegeneration. For purposes of this invention, beneficial or desired clinical results include, without limitation, an alleviation of one or more clinical indications, decreased motor neuron degeneration, reduced severity of one or more clinical indications, diminishment of the extent of disease, stabilization of the disease state (i.e., not worsening), delay or slowing, halting, or reversing neurodegenerative disease progression, and partial or complete remission, whether detectable or undetectable. “Treatment” also refers to prolonging survival by weeks, months, or years as compared to expected survival if not receiving treatment according to a method provided herein. Subjects in need of treatment can include those already having or diagnosed with a neurodegenerative condition or disorder as well as those prone to, likely to develop, or suspected of having the neurodegenerative condition.

As used herein, the term “neurodegenerative disease” refers to a disease or disorder affecting nerves of the central nervous system that typically manifests as one or a combination of motor, sensory, sensorimotor, or autonomic neural dysfunction. A neurodegenerative disease can be sporadic or genetically acquired, can result from systemic disease or traumatic injury, or can be induced by a neurotoxic agent such as a chemotherapeutic agent. In exemplary embodiments, a neurodegenerative disease appropriate for the present invention is one associated with or characterized, at least in part, by neurofilament (NF) aggregation and neurite degeneration. Neurodegenerative diseases appropriate for the present invention include, without limitation, Parkinson's disease (PD), Alzheimer's disease (AD), Huntington's disease (HD), Spinal Muscle Atrophy (SMA), spinocerebellar ataxias (SCA), Guillain-Barré syndrome (GBS), Parkinson's disease-related disorders, Charcot-Marie-Tooth disease (CMT), amyotrophic lateral sclerosis (ALS), and other motor neuron diseases.

Huntington's Disease (HD) is characterized by progressive neuronal death in different areas of the brain and the appearance of motor coordination deficits and hyperkinetic movement disorders. The spinocerebellar ataxias (SCA) are a group of neurodegenerative diseases characterized by cerebellar ataxia (deficit or failure of muscular coordination), occulomotor abnormalities, upper and lower motor neuron signs, cognitive decline, epilepsy, autonomic dysfunction, sensory deficits, and psychiatric manifestations. See, e.g., Schols et al., Lancet Neurol 3(5):291-304 (2004). Charcot-Marie-Tooth disease (CMT) (also known as Hereditary Motor and Sensory Neuropathy, “HMSN”) is the most common inherited disorder of the peripheral nervous system, affecting approximately 1 in 2500 individuals. Clinical subtypes of CMT are associated with progressive distal muscle weakness, atrophy, demyelinating neuropathy (CMT type 1), and axonal loss (CMT type 2).

Amyotrophic lateral sclerosis (ALS) is the most common motor neuron (MN) disease having no effective treatment (Robberecht and Philips, Nat. Rev. Neurosci. 14:248-264 (2013)). A clinical diagnosis of ALS, defined by progressive signs and symptoms of upper and lower motor neuron dysfunction, is typically confirmed using electromyography. Additional tests such as magnetic resonance imaging (MRI) of the brain or spinal column can exclude other diseases. In the absence of treatments to effectively delay or halt disease progression, ALS patient care is largely palliative. While mostly sporadic, some cases of ALS are associated with genetic mutations, among which 20% is caused by mutations in the copper zinc superoxide dismutase (SOD1) gene.

In some cases, the present invention provides a gene therapy method for treating a neurodegenerative disease in a subject in need thereof, where the neurodegenerative disease affects motor neurons or is associated with motor neuron degeneration. The primary goal of gene therapy is to treat a loss-of-function genetic disorder by delivering correcting therapeutic DNA sequences into the nucleus of a cell, whereby long-term expression of such therapeutic DNA sequences at physiologically relevant levels can partially or fully correct the loss-of-function phenotype. Therapeutic gene transfer offers potential advantages over direct administration of a polypeptide systemically or via continuous or targeted production of a target gene product in vivo. Embodiments of the invention comprise delivery of a gene therapy vector having a heterologous gene of interest to obtain stable gene expression in target cells or tissues. The method can include contacting a cell or tissue of a subject in need thereof to a vector comprising a heterologous gene, wherein the vector is introduced into a motor neuron of the subject and wherein expression of the heterologous gene treats the neurodegenerative disease. In some cases, the subject has been diagnosed or is suspected of having amyotrophic lateral sclerosis (ALS) or spinal muscular atrophy of infancy.

As used herein, the phrase “expression of the heterologous gene” refers to expression of a therapeutically effective amount of a heterologous gene in a cell or cells to which the gene therapy vector has been introduced. The “heterologous” gene may be a second copy or an altered copy of a gene that is already part of the subject's genome. Continuous in situ production of physiological concentration of the gene product can provide a therapeutically effective amount of such molecules, thereby treating the neurodegenerative disease. In some cases, the heterologous gene encodes NF-68(L) protein (“NF-L”). Nucleotide and amino acid sequences for NF-L are set forth as SEQ ID NO: 1 and SEQ ID NO:2, respectively. In such cases, introducing a viral gene therapy vector comprising nucleic acid sequence encoding NF-L polypeptide, and targeting the vector to one or more types of neurons, can increase expression of NF-L mRNA and the proportion of NF-L subunit to relative to subunits NF-200(H) (“NF-H”) and NF-145(M) (“NF-M”), whereby the neurodegenerative disease is treated. Nucleotide and amino acid sequences for NF-H are set forth as SEQ ID NO:3 and SEQ ID NO:4, respectively. Nucleotide and amino acid sequences for NF-M are set forth as SEQ ID NO:5 and SEQ ID NO: 6, respectively.

Targeted delivery of a therapeutically effective amount of a heterologous gene can treat, prevent, reverse, remove, or compensate for NF aggregation or neurite degeneration in susceptible motor neurons. Accordingly, targeted delivery of a therapeutic gene such as NF-L to vulnerable neurons of the central nervous system or peripheral nervous system can be a useful neuroprotective (e.g., prophylactic) or therapeutic strategy for a subject having, suspected of having, predisposed to developing, or likely to be susceptible to a neurodegenerative disease.

In exemplary embodiments, recombinant nucleic acid sequences are administered in a vector. Vectors appropriate for use according to a method of the present invention include, without limitation, viral vectors, preferably adenoviruses, herpes viruses, adeno-associated viruses (AAV), retroviruses including lentiviruses, poxviridae, baculovirus, vaccinia or Epstein-Barr viruses. In exemplary embodiments, vectors are adenoviruses. Various serotypes of adenovirus are known in the art. For example, an appropriate adenovirus serotype for use according to a method of the present invention can be a type 2 or type 5 human adenovirus (Ad2 or Ad5) or an adenovirus of animal origin (e.g., adenoviruses of bovine, canine, murine, ovine, porcine, avian, and simian origin). In some cases, it is preferable to use replication-defective adenoviruses, comprising at least one non-functional viral region selected from E1, E2 and/or E4. Such adenoviruses may be produced according to conventional methods such as the method described by Dedieu et al., J. Virology 71:4626-4637 (1997). Recombinant adenoviruses carrying a replication defective genome can be prepared according to methods known in the art using either competent packaging cells or transient transfection (Graham F. L. and Prevec L., Gene transfer and expression protocols; Manipulation of adenovirus vectors, Methods in Molecular Biology (1991), The Humana Press Inc, Cliften, NJ, chapter 11, pp. 109-128). In some cases, a method of treating a neurodegenerative disease in a subject in need thereof comprises contacting a cell or tissue of a subject to a recombinant adeno-associated virus vector comprising a heterologous gene, wherein the vector is introduced into a neuron (e.g., motor neuron) of the subject and wherein expression of the heterologous gene treats the neurodegenerative disease.

In another aspect, the present invention provides a method of protecting against axonal or neurite degeneration in a subject in need thereof. The method comprises administering one or more recombinant nucleic acid sequences (polynucleotides) encoding a NF-L polypeptide to tissue of the subject, wherein the nucleic acid sequences are targeted to, for example, neurons of the tissue (e.g., motor neurons), and wherein expression of NF-L in targeted neurons protects the motor neurons from neurite degeneration. In some cases, the subject has been diagnosed or is suspected of having ALS or spinal muscular atrophy of infancy. Recombinant nucleic acid sequences can be administered in a vector such as a viral vector or virus-derived vector. Viral vectors appropriate for use according to a method provided herein include, without limitation, adenoviruses, retroviruses, herpes viruses, and adeno-associated viruses. In some cases, a motor neuron-specific promoter can be used to drive expression of a therapeutic target gene such as NF-L. The efficacy of gene therapy may be monitored by clinical assessment as well as measurement of phosphorylated neurofilament levels as reduction of axonal (neurite) degeneration will decrease the release of phosphorylated neurofilament.

b. Diagnostic Methods

In another aspect, the present invention provides a method of diagnosing a subject as having a neurodegenerative disease. The method can comprise detecting neurofilament protein aggregation, neurite degeneration, and/or cell death as an indicator of neurodegenerative disease in the subject. For example, a method of the present invention can comprise (a) obtaining induced pluripotent stem (iPS) cells from somatic cells of a subject, wherein the iPS cells are capable of differentiation into motor neurons; (b) culturing the iPS cells under conditions suitable to differentiate the iPS cells into motor neurons; and (c) detecting neurofilament levels and aggregation or neurite degeneration in the iPS cell-derived motor neurons, where reduced neurofilament levels, or neurofilament aggregation or neurite degeneration indicates that the subject has a neurodegenerative disease.

In exemplary embodiments, the subject is a living human. For example, the subject can be a living human suspected of or at risk for developing a neurodegenerative disorder. Skin biopsy or blood samples can be obtained from such individuals and reprogrammed into motor neurons either directly (Vierbuchen et al., Nature 463:1035-1041; Son et al., Cell Stem Cell 9:205-218) or indirectly via iPS cells as exemplified herein. An individual's (e.g., human patient) motor neurons can be assayed for reduced levels of NF-L polypeptide or mRNA, increased release of phosphorylated neurofilament to culture media, or neurofilament aggregation, neurite degeneration, susceptibility to stress, or increased cell death. NF-L levels can be assayed using, for example, RT-PCR (for mRNA) or using an anti-NF-L antibody (e.g., Western blot). Release of soluble phosphorylated NF into culture medium can be detected using, for example, an enzyme-linked immunosorbent assay (ELISA). Such assays could be readily performed in a clinical setting. In some cases, diagnosis of an individual suspected of or at risk for developing a neurodegenerative disorder may be validated by detecting increased levels of phosphorylated neurofilament in a cerebral spinal fluid sample obtained from the individual.

c. Screening Methods

In another aspect, the present invention provides methods for identifying candidate therapeutic agents to treat a neurodegenerative disease, to slow or halt neurodegeneration, to alter a neurodegenerative disease mechanism, or to correct an observed neurodegenerative disease phenotype. For example, methods of the present invention can comprise testing compounds for their ability to modify or restore cellular levels of NF-L, to restore cellular proportions of NF subunits, or to attenuate or prevent neurite degeneration. Alternatively, the methods provided herein could identify compounds that promote cell survival (e.g., compounds that stimulate intracellular protective pathways or promote secretion of growth factors) independent of NF function.

In some cases, the present invention provides a method of evaluating a candidate neuroprotective agent, where the method comprises the steps of contacting a candidate neuroprotective agent to motor neurons derived from induced pluripotent stem (iPS) cells obtained from somatic cells of a human amyotrophic lateral sclerosis (ALS) patient, wherein the motor neurons exhibit a phenotype typical of ALS; and evaluating the contacted motor neurons for a neuroprotective effect of the agent. In some embodiments, the method will include evaluating the effect of the agent relative to motor neurons derived from iPS cells obtained from somatic cells of a human ALS patient that have not contacted the agent. A “neuroprotective effect” can include, without limitation, a reduction in severity of neurodegeneration, a delay in onset of neurodegeneration, a reduction in severity of neurofilament (NF) aggregation and neurite degeneration, and increased motor neuron viability in vitro. For example, a compound contacted to an ALS iPS-derived motor neuron culture can alter neurofilament levels, neurofilament subunit proportion, neurofilament aggregation, axonal or neurite degeneration, or cell survival and such effects of the compound can be assayed, selected, and validated. See, for example, Yang et al., Cell Stem Cell. (2013) Boyd et al., J Biomol. Screen. 19(1):44-56 (2014); Naohiro et al., Sci. Transl. Med. 145:104 (2012); Sharma et al., Methods Enzymol. 506:331-60 (2012); Burkhardt et al., Mol. Cell Neurosci. 56:355-64 (2013).

In some cases, the method can comprise differentiating iPS cells, derived from somatic cells of a human subject, into neurons, preferably as disclosed below, and examining the effect of a test compound on NF subunit proportions or NF-L levels of the motor neurons, where an increase in NF-L protein relative to motor neurons derived from iPS cells from somatic cells of an ALS patient that have not been exposed to the test compound indicates that the compound modifies cellular NF subunit proportions or NF-L levels. As used herein, the phrases “differentiation into neurons” and “differentiating into neurons” refer to promoting the differentiation of iPS cells into cells having the genetic markers, cell function, and cell morphology characteristic of a particular neuronal lineage (e.g., motor neurons, Gabaergic neurons, cholinergic neurons, dopaminergic neurons).

The method can additionally or alternatively comprise examining the effect of a test compound on axonal or neurite degeneration relative to neurons derived from iPS cells from somatic cells of a human subject having a neurodegenerative disease that have not been exposed to the test compound. In some cases, the method comprises examining the effect of a test compound on axonal or neurite degeneration relative to motor neurons derived from iPS cells of a human ALS patient that have not been exposed to the test compound. Such effects on ALS-patient derived motor neurons can be detected by assaying for NF-L protein production, NF-L mRNA levels, degree of neurite degeneration, and onset or extent of cell death. Changes in neurite length are detected as an indicator of neurite degeneration. In exemplary embodiments, neurite length is measured using a reporter system such as the luciferase reporter NanoLuc (Nluc) fused with SYNAPTOPHYSIN (SYP), a synaptic glycoprotein that targets the Nluc reporter to axonal membrane, as described in U.S. Patent Application Ser. No. 62/112,441, filed Feb. 5, 2015 (incorporated herein by reference in its entirety).

In another embodiment, the method includes screening test compounds for an effect on motor neuron survival. Compounds that increase motor neuron cell survival, relative to ALS-patient derived motor neurons that have not been exposed to the test compound, are excellent candidates for further drug testing.

In some cases, the method further comprises obtaining a population of iPS cells derived from somatic cells from human subject known to have a neurodegenerative disease such as ALS. In exemplary embodiments, the somatic cells of an ALS patient are fibroblasts. Other types of somatic cells may also be used, including without limitation, blood cells, hair follicle cells, fat cells, and neural cells. To confirm that reprogramming of wild-type and ALS fibroblasts to a pluripotent state has occurred, one may wish to use standard techniques including, but not limited to, quantitative PCR with reverse transcription (qRT-PCR), teratoma formation, DNA fingerprinting and microarray analysis. Pluripotency criteria have been described (see Chan et al., Live cell imaging distinguishes bona fide human iPS cells from partially reprogrammed cells, Nat. Biotech., 27:1033-1037, 2009). Typically, one would look for teratoma formation and expression of endogenous Oct4, SSEA, TRA, and other markers. One could also assay for the ability of the cells to make embryoid bodies that produce all three dermal lineages.

### Compositions of the Invention

In a further aspect, the present invention provides compositions useful for treating a neurodegenerative disease or protecting against neurite degeneration in a subject in need thereof. In particular, the present invention provides recombinant nucleic acid molecules and expression vectors comprising such nucleic acid molecules. In some cases, a recombinant nucleic acid molecule comprises a nucleic acid sequence encoding a NF-L polypeptide (e.g., human NF-L). A recombinant nucleic acid molecule of the invention can further comprise a motor neuron-specific promoter operably linked to a target nucleic acid sequence (e.g., a nucleic acid sequence encoding human NF-L).

In exemplary embodiments, a recombinant nucleic acid molecule of the present invention is in a vector. In some cases, the vector is a plasmid (e.g., plasmid expression vector) or is a virus or virus-derived. For example, the vector can be a virus selected from the following: an adenovirus, a retrovirus, a herpes virus, and an adeno-associated virus. In some cases, the vector is a replication-defective adenovirus vector (e.g., a human replication-defective adenovirus). A replication-defective adenovirus vector is capable of delivering its genome to an infected cell, but comprises one or more disabling mutations that prevent activation of viral early gene expression and DNA replication. For example, the replication-defective adenovirus vector pAdRSVβgal lacks the early region 1 (E1) genes needed to efficiently activate transcription of the other viral early genes, including those encoding viral DNA replication proteins and those responsible for inactivating the cellular DNA damage response. See Stratford-Perncaudet et al., J. Clin. Invest. 90:626-30 (1992).

In another aspect, the present invention provides kits useful for the diagnosis, treatment, or monitoring of a neurodegenerative disease. For example, a kit of the present invention can be used to diagnose or monitor disease progression in a subject predisposed to or suspected of developing a neurodegenerative disease or suffering from a neurodegenerative disease. In some cases, a kit of the present invention can comprise the following components: at least one oligonucleotide primer capable of hybridizing to or amplifying a target NF-L nucleic acid sequence; at least one reference corresponding to a level of NF-L target nucleic acid; at least one buffer or reagent; and a container. By way of example, oligonucleotide primers appropriate for a kit of the present invention include, without limitation, the nucleotide sequences of SEQ ID NO:7 (5′-ATGAGTTCCTTCAGCTACGAGC-3′) and SEQ ID NO:8 (5′-CTGGGCATCAACGATCCAGA-3′).

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents and alternatives falling within the spirit and scope of the invention as defined by the appended claims.

The invention will be more fully understood upon consideration of the following non-limiting Examples.

### EXAMPLES

### Example 1—Efficient Differentiation of ALS iPS Cells and Genetically Corrected ALS iPS Cells to Motor Neurons

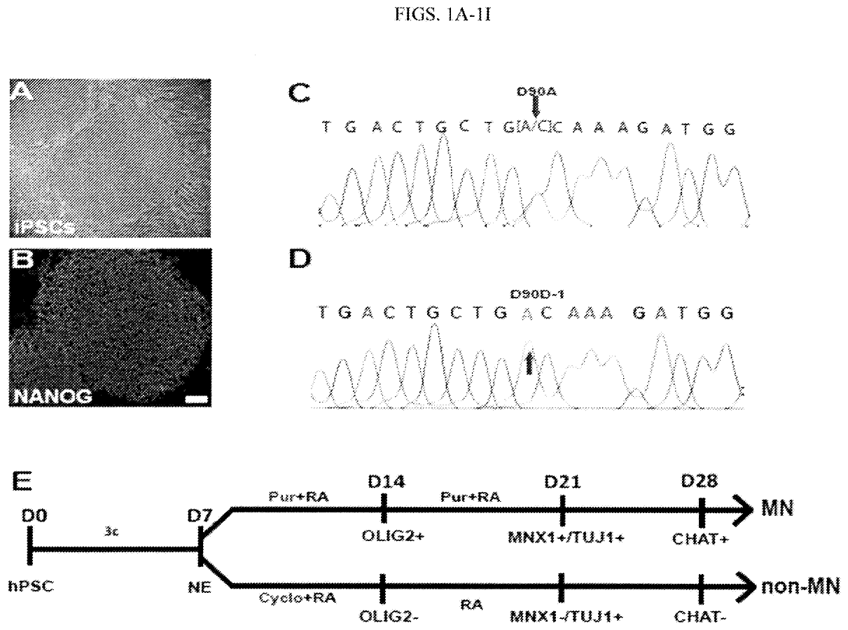

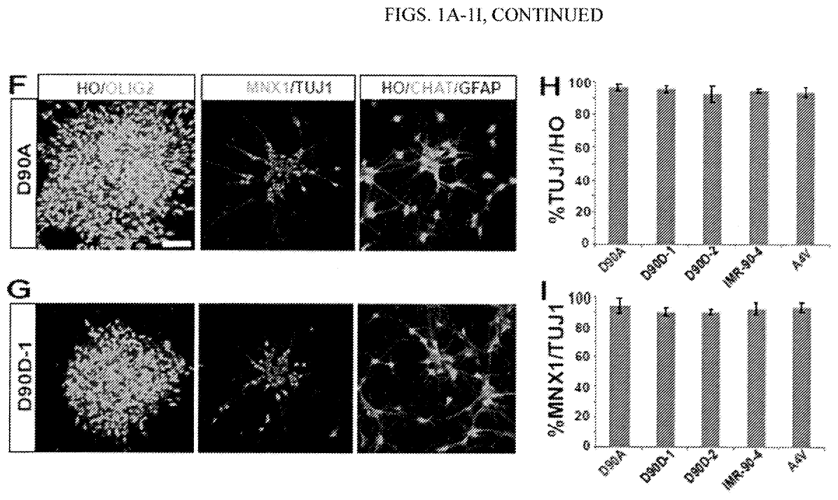

Fibroblasts from a 50-year old female carrying the D90A SOD1 mutation (ND29149, P3; Coriell Institute) were reprogrammed using the non-integrating Sendai virus as described (Ban et al., Proc. Natl. Acad. Sci. U.S.A 108:14234-14239 (2011)). A4V SOD1 mutant iPSC lines, established with retrovirus, were obtained from Coriell (ND35671). The IMR-90-4 iPSC line, generated from fetal fibroblasts (Hu et al., 2010), was used as a wild-type (wt) control. The iPS cells reprogrammed with Sendai virus were integration-free as confirmed by qPCR analysis for viral sequences (data not shown). All iPS cells became stable cell lines, exhibited typical morphology, expressed the pluripotency markers including NANOG (FIGS. 1A-B), OCT4, SSEA-4, and SOX2, generated teratomas in vivo, and retained karyotype stability (some data not shown). Human ESCs (H9 line, NIH registry 0046) were used as an additional wt control and as a recipient for transgenic expression of mutant SOD1. The SOD1 D90A mutation was maintained as confirmed by sequencing exon 4 of SOD1 (FIG. 1C, 1D).

To establish isogenic controls under the same genetic and epigenetic background, we corrected the D90A mutation in the iPS cells using TALEN mediated homologous recombination. Transgenes were targeted to the AAVS1 locus in hESCs and iPSCs by TALEN. The donor plasmid for constitutive expression of D90A SOD1 was constructed by replacing the CAG-GFP cassette of the plasmid AAV-CAGGS-EGFP (Addgene, Cambridge, MA) with the CAG-D90A SOD1 cassette via SpeI and MluI double digestion. Similarly, the Tet-inducible expression plasmid was constructed by replacing the CAG-GFP cassette with the CAG-Tet-On 3G cassette (pTRE3G-SV40-polyA cassette, Clontech, Mountain View, CA) via the SpeI site. NF-L cDNA sequence was inserted into the SalI and MluI sites of the plasmid for conditional NF-L expression. To correct the SOD1 D90A mutation, a 986 basepair (bp) or 829 bp fragment at both sides of the TALEN target point was PCR-amplified using genomic DNA from H9 hESCs. The digested fragments were then cloned into the multiple clone site of plasmid PL452 (Frederick National Lab). TALENs pairs targeting the AAVS1 locus was designed as described (Hockemeyer et al., Nat. Biotechnol. 29:731-734 (2011)). TALEN activity was assayed via surveyor nuclease (Transgenomic, Omaha, NE). A standard protocol was used for cellular transfection and cloning as described (Hockemeyer et al., Nat. Biotechnol. 29:731-734 (2011)).

PCR analysis of individual single-cell-derived G418-resistant clones using a primer external to the 5′ donor homology region and a primer against the PGK promoter demonstrated disruption of the genomic locus and integration of the donor vector with a frequency of about 45% (data not shown). Two selected clones, designated as D90D1 and D90D2, showed successful correction of the targeted locus, as revealed by TaqMan SNP genotyping assays and by sequencing (FIG. 1C, 1D). The corrected iPSCs displayed a normal karyotype, uniform expression of pluripotency markers, and generation of teratomas, and identical polymorphisms to their parental cells as assayed by short tandem repeat analysis.

Pluripotent stem cells were first differentiated to neuroepithelia in a neural medium consisting of DMEM/F12, N2 supplement, and non-essential amino acids in the presence of SB431542 (2 μM), LDN193189 (300 nM), and CHIR99021 (3 μM; Stemgent) for 7 days. At day 8, the neuroepithelia were treated with RA (0.1 μM) and purmorphamine (0.5 μM) for 7 days for MN induction. For generation of non-MNs, cyclopamine (0.5 μM) was added in place of purmorphamine. At day−14, both MN and non-MN progenitors in the form of rosettes were isolated and expanded as floating clusters in suspension in the same respective medium but without SB431542, LDN193189, and CHIR99021 for an additional 7 days before plating on laminin substrate for generating mature neurons. To generate synchronized post-mitotic neurons, the cultures were treated from day 18-21 with compound E (0.1 μM) to block cell proliferation.

Initial experiments were designed to assay whether MN generation is altered by SOD1 mutations. Using a protocol (FIG. 1E) modified from previous methods (Amoroso et al., J. Neurosci. 33:574-586 (2013)); Li et al., Stem Cells 26:886-893 (2008)), it was determined that both the mutant SOD1 iPSCs (D90A and A4V) and genetically corrected (D90D), as well as wt iPSCs (IMR-90-4), efficiently differentiated into OLIG2⁺ MN progenitors at day 14, MNX1⁺ postmitotic MNs at day 21, and CHAT⁺ maturing MNs by 21-30 days (FIGS. 1F-G), with 94% of the TUJ1⁺ neurons, or 90% of total differentiated cells being MNX1⁺ MNs (FIG. 1H-I). These results indicated that ALS mutations do not affect MN development. We also differentiated the iPSCs to spinal neurons that were void of MNX1⁺ or CHAT⁺ MNs by blocking hedgehog signaling using cyclopamine during neural patterning (FIG. 1E), which forms a non-MN control within the individual iPSC line. At 4-5 weeks of iPSC differentiation, no glial fibrillary acidic protein (GFAP)-expressing astrocytes were observed (FIG. 1F-G), which is a similar result to previous observations that GFAP-expressing astrocytes do not usually appear until after 2-3 months of hPSC differentiation (Krencik et al., Nat. Biotechnol. 29:528-534 (2011); Serio et al., Proc. Natl. Acad. Sci. U.S.A 110:4697-4702 (2013)). Additionally, we used compound E, a notch signaling inhibitor, to prevent generation of new neurons from progenitors. The highly enriched and synchronized MNs and non-MNs enable phenotypic characterization at cellular and molecular levels.

### Example 2—ALS Motor Neurons Exhibit Small Aggregates of Mutant SOD1 and NE Aggregates

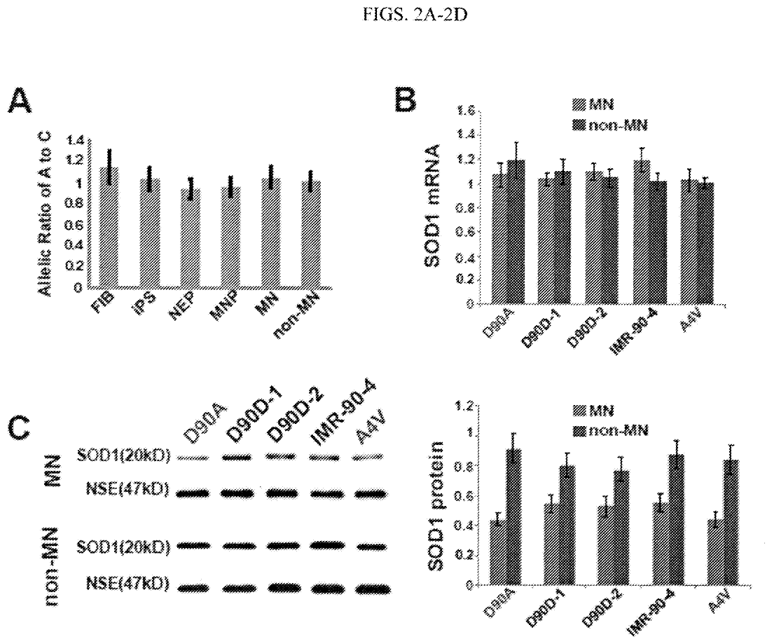

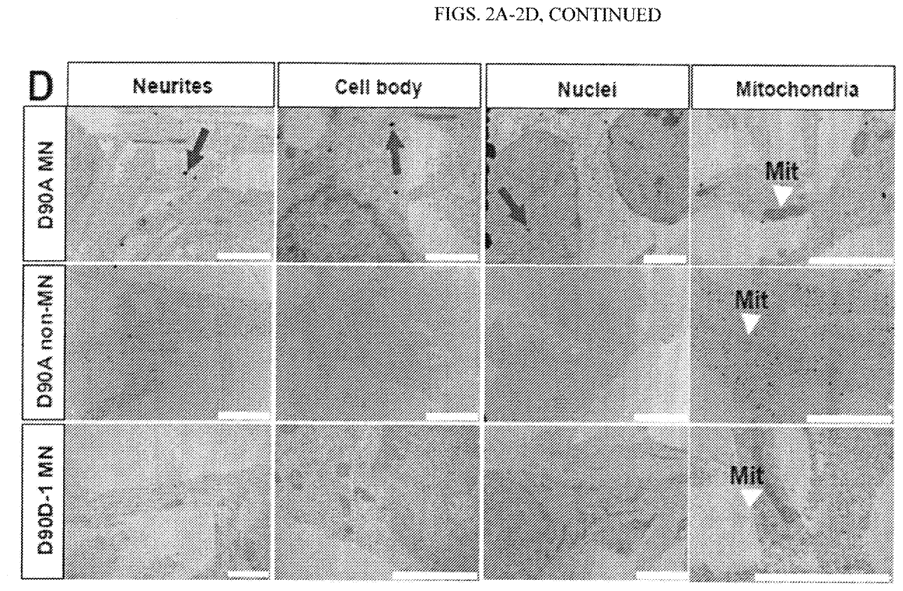

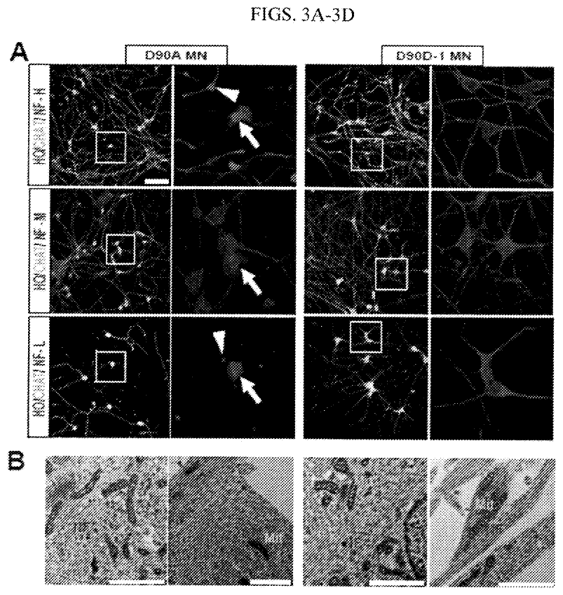

Expression of multiple copies of disease-causing mutant SOD1 in animals results in increased expression and aggregation of SOD1 (Bruijn et al., Science 281:1851-1854 (1998); Furukawa et al., Proc. Natl. Acad. Sci. U.S.A 103:7148-7153 (2006); Karch et al., Proc. Natl. Acad. Sci. U.S.A 106:7774-9 (2009)). We first asked if SOD1 is comparably expressed in MNs and non-MNs. Western blotting indicated that the SOD1 level in day-30 MNs was approximately 40% lower than that in non-MNs (FIG. 2C). Next, we assayed whether a single copy of mutant SOD1 would alter the level of SOD1 and cause its aggregation in human ALS neurons (neurons of human subjects having ALS). By quantitative PCR (qPCR), it was observed that the ratios between the mutant and wild-type (wt) alleles were about 1 in fibroblasts, iPSCs, neuroepithelia, MN progenitors, MNs, and non-MNs for the D90A mutant, whereas in wt cells both copies were wt (FIG. 2A), indicating that the mutant SOD1 copy is maintained during reprogramming and neural differentiation. By RT-qPCR we then found that mutant SOD1 MNs and non-MNs expressed a similar SOD1 level as wt and genetically corrected ALS cells (FIG. 2B). Interestingly, Western blotting indicated that mutant MNs, but not mutant non-MNs, expressed an even lower level of SOD1 than wt and genetically corrected ALS MNs (FIG. 2C). This unexpected pattern of changes was replicated in six sets of biological samples. Thus, unlike in transgenic animals, the total amount SOD1 protein does not increase in ALS patients' MNs, at least at the age analyzed.

Immunostaining for SOD1 and examination under confocal microscopy showed a ubiquitous expression pattern without discernible aggregates in MNs. We reasoned that aggregates may have been absent or too small to discern under light microscopy. Immuno-electron microscopy (EM) revealed an even distribution of fine gold particles in cytoplasm and neurites of MN cultures from wt and genetically corrected ALS cells. In the ALS MN cultures, clusters of gold particles, averaged 64±5 nm in diameter, were present in cytoplasm, neurites, and nuclei (FIG. 2D). In transgenic animals, SOD1 aggregates are often present on mitochondrial membrane (Bergemalm et al., J. Neurosci. 26:4147-4154 (2006); Pasinelli et al., Neuron 43:19-30 (2004); Vijayvergiya et al., J. Neurosci. 25:2463-2470 (2005)). Careful examination revealed no association of SOD1 aggregates with mitochondria in ALS patients' MNs (FIG. 2D). In the ALS non-MN cultures, there were more gold particles than in MNs, but they were singular, of 10.73±1.07 nm in diameter (FIG. 2D, the fine particles were revealed in non-contrasted image for non-MNs). Thus, small SOD1 aggregates are present in the cytoplasm, nuclei, and neurites but not in mitochondria of ALS MNs.

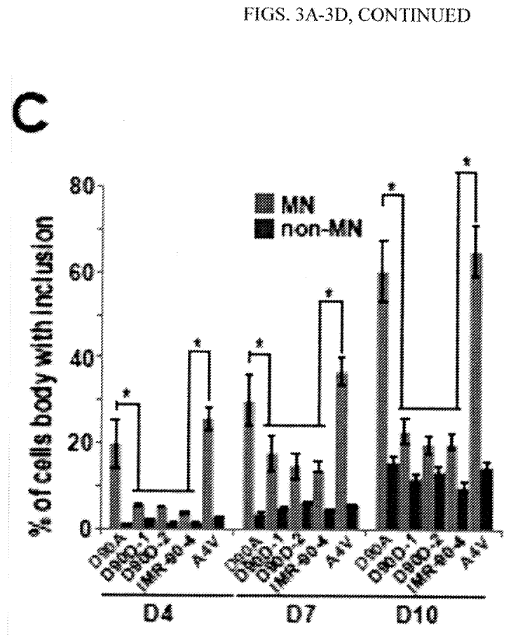

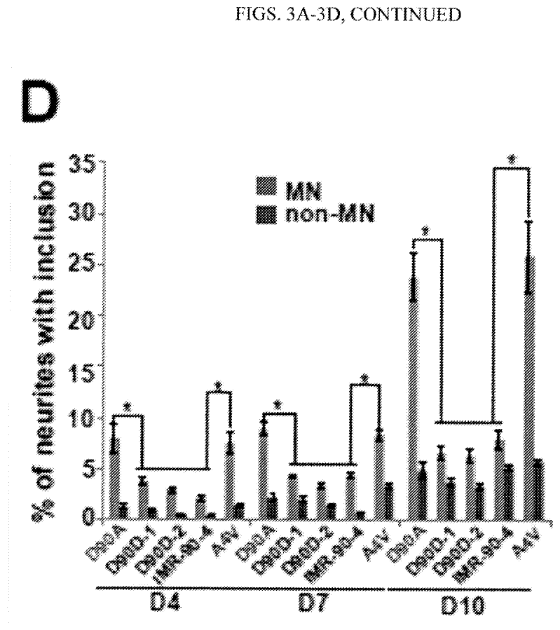

NF aggregates in the perikaryon and proximal axons of spinal MNs is hallmark ALS pathology (Carpenter, Neurology 18:841-851 (1968); Hirano et al., J. Neuropathol. Exp. Neurol. 43:471-480 (1984)). Immunostaining for NF-200(H), NF-145(M) and NF-68(L) at day−30 revealed distinct, focal accumulation of immunoreactive products in cytoplasm and neurites of CHAT⁺ ALS MNs (FIG. 3A). Such NF aggregates were rare in ALS non-MNs or MNs from wt and genetically corrected ALS iPSCs (see FIG. 3A). The aggregates in the cytoplasm and proximal neurites were often accompanied by lower immunofluorescent staining for NF (FIG. 3A) and other proteins (CHAT) in distal neurites. However, the distribution of βIII-tubulin in ALS MN neurites was not altered. The identity of the aggregates was confirmed by electron microscopy, showing disorganized NFs near the nucleus or in the proximal neurite with mitochondria surrounding or inside of the aggregate (FIG. 3B).

NF aggregates were defined as a focal accumulation of immunoreactive products with its intensity being 3 times higher than that in its surroundings. Neurite swelling or beading was defined as an enlargement of a neurite that is at least twice the diameter of the neurite. At least 500 neurites were counted in each group. Statistical analyses were performed using one-way ANOVA (Tukey or Dunnett for multiple comparisons) in SPSS13.0. Quantification of NF aggregates indicated an increasing number of ALS MNs and their neurites that contained NF aggregates at day−24, 27, and 30, or 4, 7, and 10 days after plating day−21 cells for maturation (FIGS. 3C-D). By day-10, over 60% of the MN cell bodies or 25% of neurites contained NF aggregates and the average size of the NF aggregate was 42.4˜75.4 μm² in cell bodies and 1.75˜5.53 μm² in neurites. The wt and genetically corrected ALS MNs also contained an increasing number of NF aggregates over culture but at a significantly less degree, reaching about 20% of the cells and 8% of the neurites. Fewer NF aggregates existed in non-MNs as compared to MNs in the ALS cells. Thus, NF aggregates are preferentially present in ALS MNs and over time more MNs contain aggregates.

In or surrounding the NF aggregates were often accumulation of mitochondria (FIG. 3B). In SOD1 transgenic mice, mitochondria are often swollen or contain vacuous formations (Vijayvergiya et al., J. Neurosci. 25:2463-2470 (2005); Gurney et al., Science 264:1772-1775 (1994); Wong et al., Neuron 14:1105-1116 (1995); Liu et al., Neuron 43:5-17 (2004); Bergemalm et al., J. Neurosci. 26:4147-4154 (2006)). This, however, was not observed in the ALS human MNs (FIG. 3B) despite the presence of NF aggregates.

### Example 3—Neurite Degeneration in ALS Motor Neurons

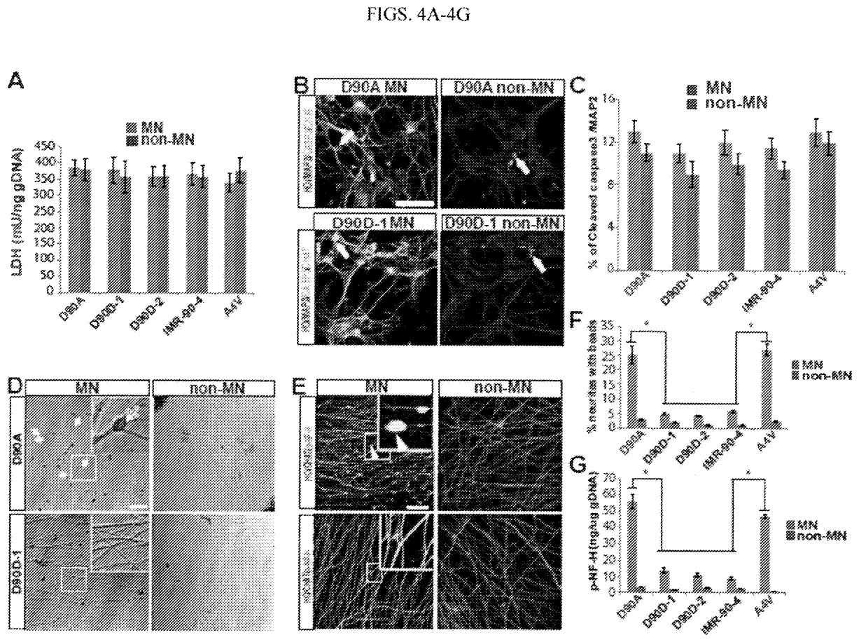

Since ALS MNs contain SOD1 aggregates and NF aggregates, we asked if these neurons undergo degeneration or cell death. Measurement of lactate dehydrogenase (LDH), a soluble enzyme located in cytosol and released during cell death, showed no obvious difference among ALS-, wt-, and corrected-MNs or non-MNs at day−10 (FIG. 4A). Immunostaining for cleaved caspase-3 showed no significant difference among the groups (FIG. 4B). These results suggest that the aggregate-containing MNs are surviving at this stage.

In ALS patients and animals, axons degenerate before symptom onset and MN death (Bruijn et al., Science 281:1851-1854 (1998); Tu et al., Proc. Natl. Acad. Sci. U.S.A 93:3155-3160 (1996); Fischer et al., Exp. Neurol. 185:232-240 (2004)). Indeed, we observed bead-like swellings along neurites of ALS MNs under phase contrast microscopy as early as day 7 after plating. At day 10, 25±2.4% neurites in ALS MN cultures exhibited bead-like structures, whereas only 5±0.4% of the neurites had such structures in control MNs (FIGS. 4C, E). Few non-MNs exhibited beading structures (FIG. 4C). Aggregated NF as well as axonal NF is often heavily phosphorylated (Goldstein et al., Proc. Natl. Acad. Sci. U.S.A 80:3101-3105 (1983); Lee et al., J. Neurosci. 6:850-858 (1986)). Plasma phosphorylated neurafilament H (p-NF-H) levels closely reflect disease progression and therapeutic response in the SOD1 (G93A) mice and are regarded as an ALS biomarker (Calvo et al., PLOS. One. 7: e32632 (2012)). Indeed, immunostaining for p-NF-H showed dense staining in the bead structures in neurites of the ALS MNs, giving a dotted staining appearance as opposed to an even staining pattern in control MNs or non-MNs (FIG. 4D). Similarly, CHAT staining was concentrated in the bead formations (FIG. 4D) as it was weak in other areas of the ALS MN neurites. ELISA measurement of p-NF-H in culture media indicated that its concentration in ALS MNs was 3-6 fold higher than that in the wt and corrected MNs (FIG. 4F). The p-NF-H level in non-MNs across all groups was very low (FIG. 4F). With extended cultures, we observed increased numbers of beads along the neurites and fragmentation of the neurites (not shown), suggesting neurite degeneration at a later stage. These results suggest that axons of ALS MNs undergo degeneration while the cell body is still structurally intact.

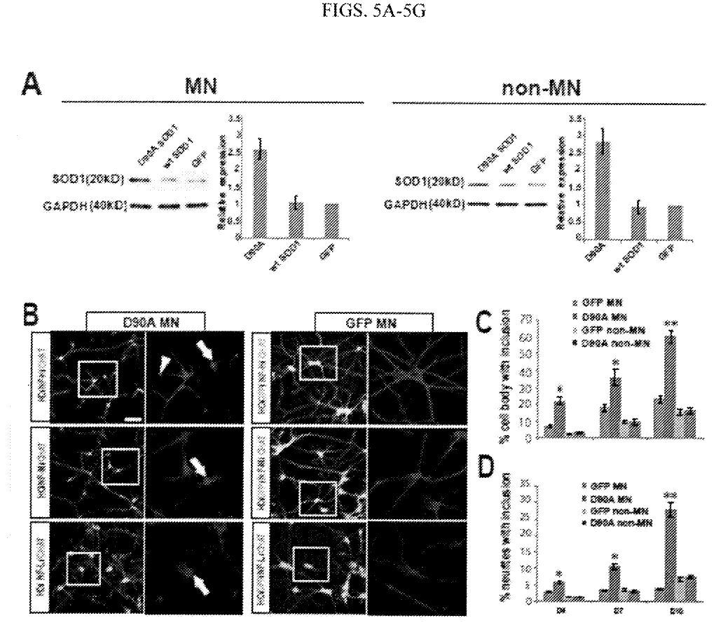

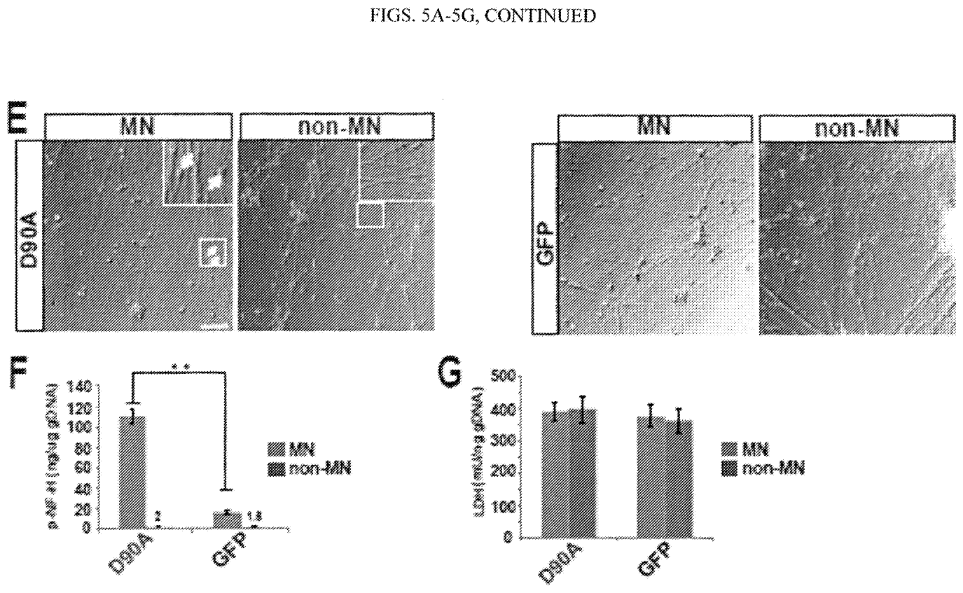

To further establish the cause/effect relationship between mutant SOD1 and the above-described motor neuron pathology, we expressed the D90A mutant SOD1 or EGFP (control) in hESCs (H9 line) in the PPP1R12C (AAVS1) locus by TALEN-mediated homologous recombination. Western blotting revealed a 2.8 fold increase in SOD1 protein in the SOD1-expressing group as compared to the EGFP control group after differentiation of the pluripotent cells to MNs and non-MNs (FIG. 5A). Expression of D90A SOD1 or EGFP did not alter the differentiation of the hESCs to MNs or non-MNs. Similar to the ALS cells, we observed progressively increased numbers of NF aggregates in both cell bodies and neurites of MNs that were derived from the D90A SOD1-expressing, but not EGFP-expressing ESCs over time (FIGS. 5B-D). However, no significant difference in NF aggregates was discerned in non-MNs between the D90A SOD1- and the EGFP-expressing groups (FIGS. 5C-D).

Analysis of neurite degeneration indicated that 90±2% of neurites presented bead-like structures in the D90A SOD1-expressing MNs whereas only 4±0.3% of the MN neurites in the EGFP-expressing group had such formations at day-10 (FIG. 5E). Few non-MNs exhibited beading structures (FIG. 5E). The p-NF-H in culture media was 6 fold higher in D90A-expressing MNs than in the EGFP-expressing MNs. By contrast, p-NF-H was barely detectable in non-MNs (FIG. 5F). Measurement of LDH in the culture media showed no significant difference among all the groups (FIG. 5G). These results indicate that expression of minimal amount of mutant SOD1 (D90A) is sufficient to cause the same disease phenotypes that are seen in cells with naturally occurring mutations.

### Example 4—ALS Motor Neurons Exhibit Altered NF Subunit Proportion

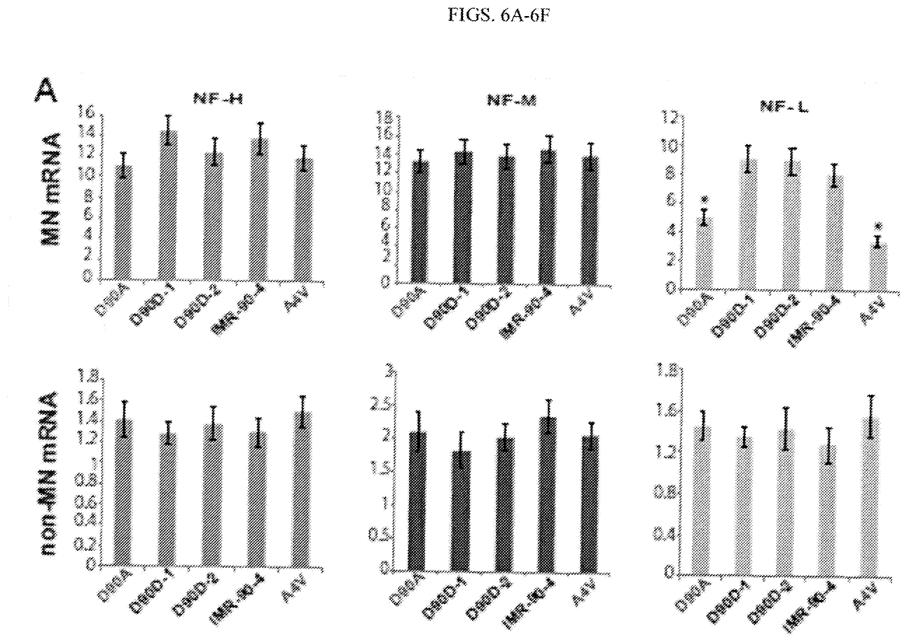

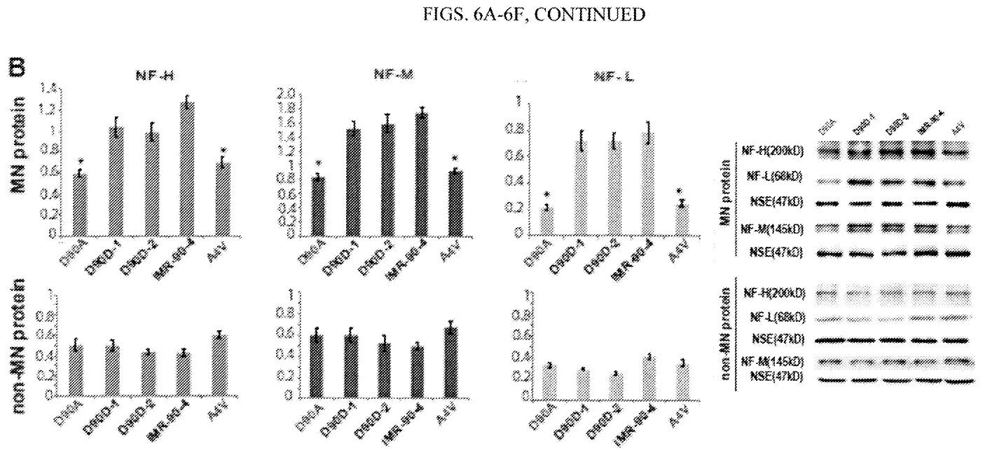

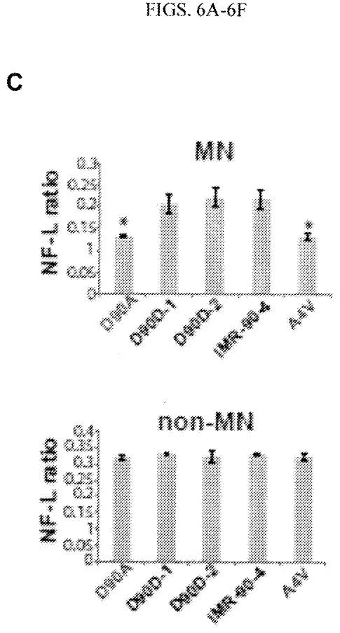

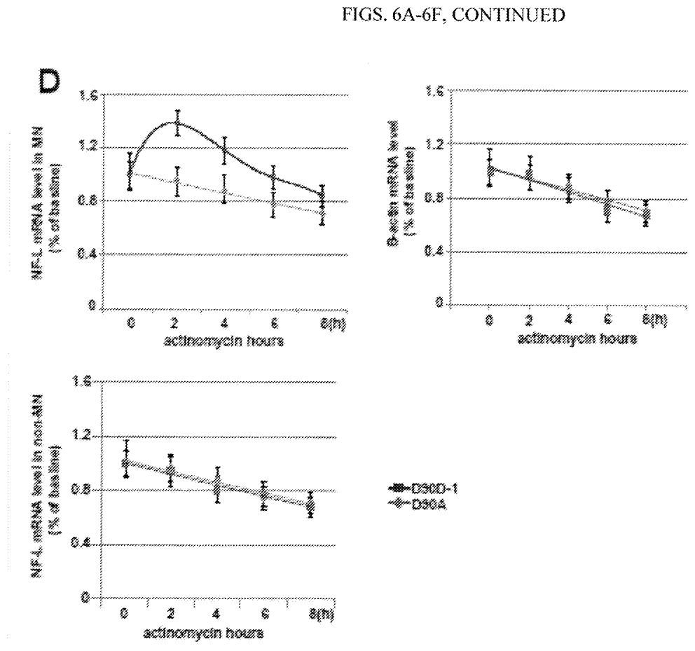

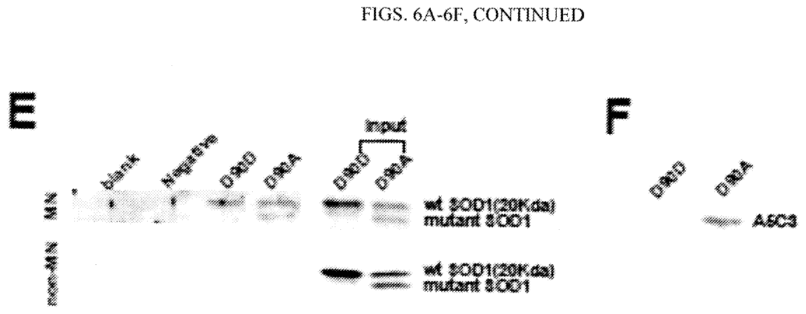

Results of the neurite degeneration assays suggest that NF aggregation is an early and key event leading to MN axonal degeneration. NFs are assembled by copolymerization of NF-L, NF-M, and NF-H in a tightly coordinated level (Hoffman and Lasek, J. Cell Biol. 66:351-366 (1975)). Transgenic disruption of their balance results in NF aggregation and MN degeneration (Cote et al., Cell 73:35-46 (1993); Xu et al., Cell 73:23-33 (1993)), resembling ALS pathology. By RT-qPCR analysis, we found no difference in the expression of NF-H and NF-M mRNA between ALS and control (wt and D90D) MNs (FIG. 6A). Interestingly, NF-L mRNA was reduced by 40˜60% in ALS MNs as compared to control MNs (FIG. 6A). In non-MNs, however, no obvious difference in all the three NF subunit mRNAs was observed between the disease and control groups (FIG. 6A).

At the protein level, Western blotting revealed that NF-H, NF-M, and NF-L were all decreased in ALS MNs but not in non-MNs as compared to control cells (FIG. 6B). Moreover, NF-L was most prominently downregulated, representing only 30% of the level in wt MNs (FIG. 6B). Because NF-L was downregulated more than the other two subunits, the proportion of NF subunits was altered in ALS MNs but not in non-MNs (FIG. 6C).

Similarly, in the MNs derived from hESCs that express the D90A SOD1, mRNA levels of NF-L, but not NF-H and NF-M, were significantly decreased as compared to controls (expressing EGFP). Strikingly, Western blotting revealed downregulation of NF subunits, especially NF-L, thus altering the proportion of NF subunits in D90A SOD1- but not EGFP-expressing MNs. In contrast, expression of mutant SOD1 or EGFP had no effect on the expression of NF subunits in non-MNs. These results further support the conclusion that mutant SOD1 alters NF compositions.

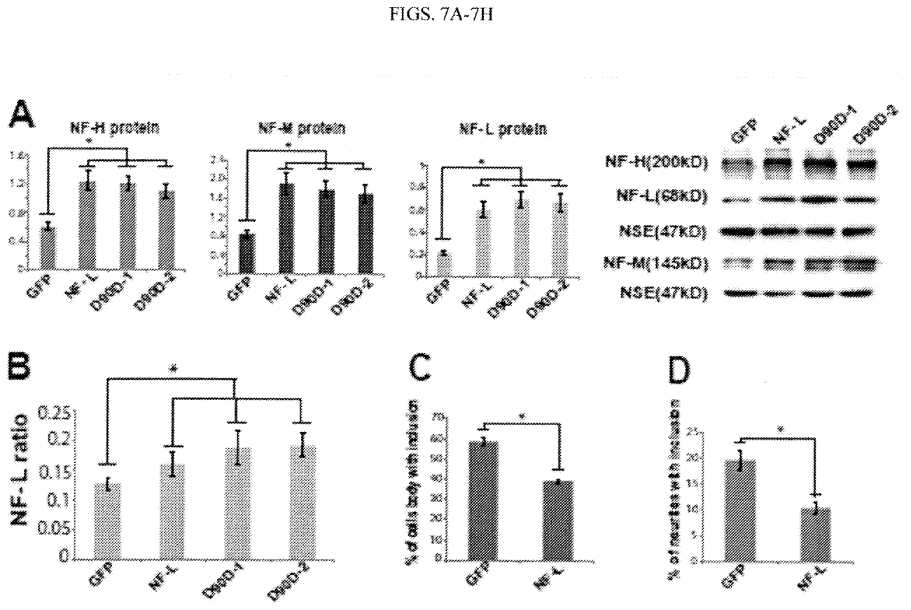

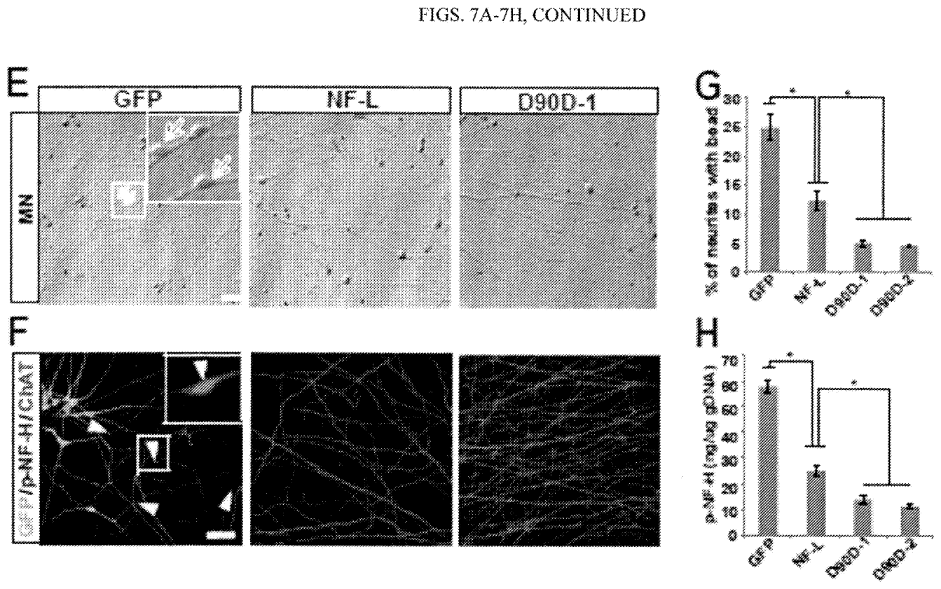

The above-described results suggest that mutant SOD1 alters the proportion of NF subunits, leading to NF aggregation and neurite degeneration. To test this hypothesis, we conditionally expressed NF-L or EGFP (control) in the PPP1R12C locus of D90A iPSCs by TALEN-mediated homologous recombination. As indicated by dose-dependent changes in EGFP intensity, the expression level of NF-L was increased in a dose-dependent manner when doxycycline (DOX) was added to the MN cultures at day-21, as shown by Western blotting. Remarkably, induction of exogenous NF-L, but not EGFP in the ALS MNs, not only increased the expression of NF-L, but also NF-H and NF-M, to the level comparable to that in corrected MNs (FIG. 7A). At 1 μg/ml DOX, the ratio of NF-L was close to that in wt or genetically corrected MNs (FIG. 7B). Importantly, the NF-L-expressing ALS MNs exhibited fewer NF aggregates in both cytoplasm and neurites at day−10 after plating when compared to the EGFP-expressing ALS MNs, with approximately 30% and 50% reductions in cytoplasm and neurites, respectively (FIGS. 7C-D). Similarly, the proportion of bead-containing MN neurites was 12% in the NF-L-expressing group as compared to 25% in the GFP-expressing group even though it was still higher than that in the genetically corrected group (FIGS. 7E, G). The p-NF-H in culture media in the NF-L-expressing ALS MNs was significantly lower than in the GFP-expressing ALS MNs (FIG. 7H). Together, these results indicate that induction of NF-L in ALS MNs largely restores NF subunit proportion, reduces NF aggregation, and mitigates neurite degeneration.

NF accumulation has been observed in ALS patients and transgenic animals (Carpenter, Neurology 18:841-851 (1968); Hirano et al., J. Neuropathol. Exp. Neurol. 43:471-480 (1984); Bruijn et al., Science 281:1851-1854 (1998); Tu et al., Proc. Natl. Acad. Sci. U.S.A. 93:3155-3160 (1996), and transgenic alteration of NFs in neurons can cause ALS-like pathology (Cote et al., Cell 73:35-46 (1993); Xu et al., Cell 73:23-33 (1993)). Such similarity has led to a hypothesis that altered stoichiometry of neuronal intermediate filaments results in ALS pathology (Julien and Kriz, Biochim. Biophys. Acta 1762:1013-1024 (2006)). Using gain-of-function (e.g., expressing mutant SOD1 in hESCs) and loss-of-function (e.g., genetic correction of D90A SOD1 mutation) analyses, we have now established unequivocally that mutant SOD1 leads to NF aggregation. The sequential appearance of NF aggregation and neurite degeneration and especially the mitigation of neurite degeneration following prevention of NF aggregation strongly suggest that NF disorganization triggers the cascade of events, leading to axonal degeneration in ALS MNs.

The key question is how NFs are disorganized in ALS MNs, leading to aggregation. It is known that over-expression of NF-H or NF-L leads to NF aggregation (Cote et al., Cell 73:35-46 (1993); Xu et al., Cell 73:23-33 (1993)), highlighting the importance of correct proportion of NF components for their physiological polymerization. In ALS patients, NF subunit proportion may be altered as there was one report showing a 60% reduction in NF-L mRNAs in MNs using in situ hybridization (Bergeron et al., J. Neuropathol. Exp. Neurol. 53:221-230 (1994)) although no information is available whether the protein levels of NF subunits are altered. Strikingly, we observed a significant and specific reduction of NF-L but not NF-H or NF-M mRNA in mutant MNs. At the protein level, all NF subunits are downregulated but the NF-L is most significantly reduced to less than one-third of the level in wild-type MNs. Thus, it is the downregulation of NFs and perhaps more importantly the altered proportion of NF subunits that result in NF aggregation. This may appear counterintuitive as MNs contain substantially higher amount of NFs than non-MNs (see FIG. 6) and it is NF over-expression that results in NF aggregation and axonal degeneration in transgenic animals (Cote et al., Cell 73:35-46 (1993); Xu et al., Cell 73:23-33 (1993)). Our present study demonstrates that when the proportion of NF subunits is restored by conditionally upregulating NF-L, NF aggregation and even neurite degeneration are significantly mitigated in ALS MNs. We, therefore, propose that alteration of NF subunit proportion in ALS MNs leads to NF aggregation, which is a critical early step toward axonal degeneration.

### Example 5—NF Aggregation and NF-L mRNA Down-Regulation in Sporadic ALS MNs

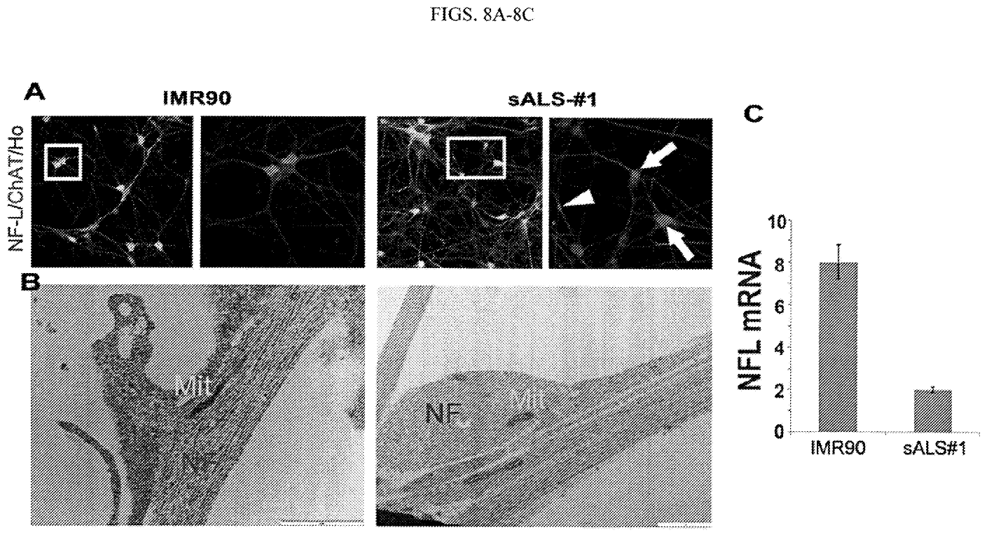

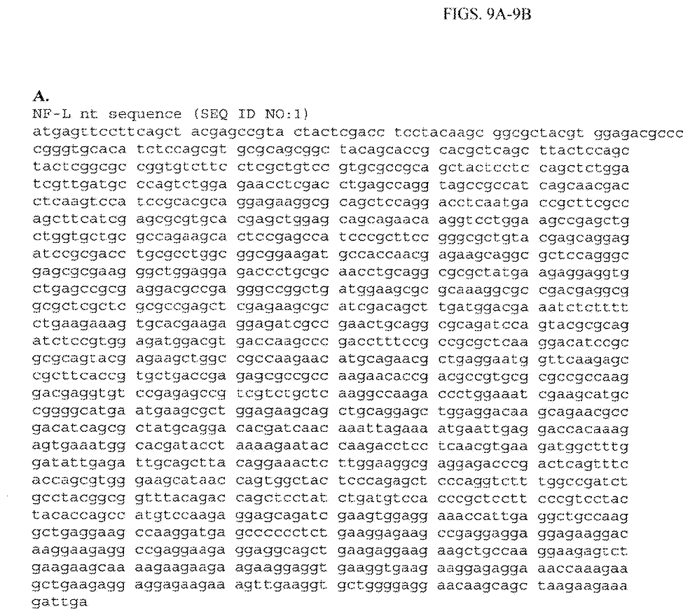

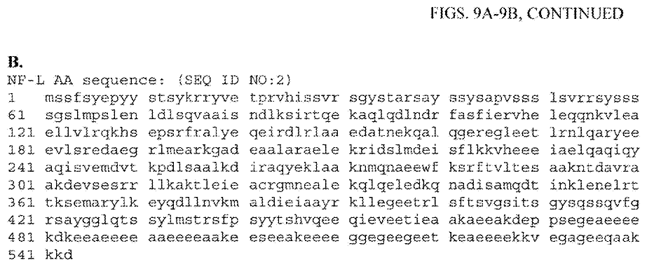

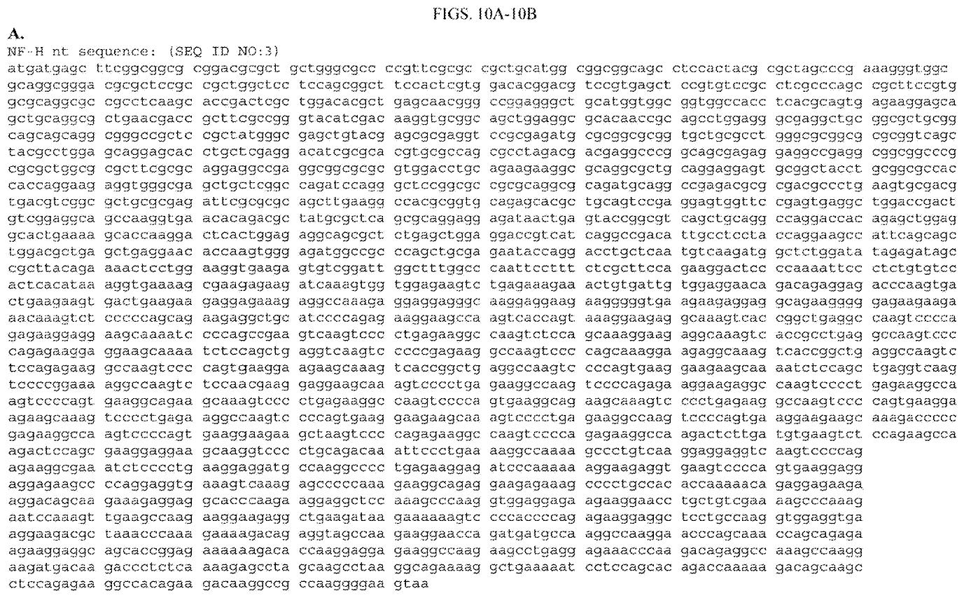

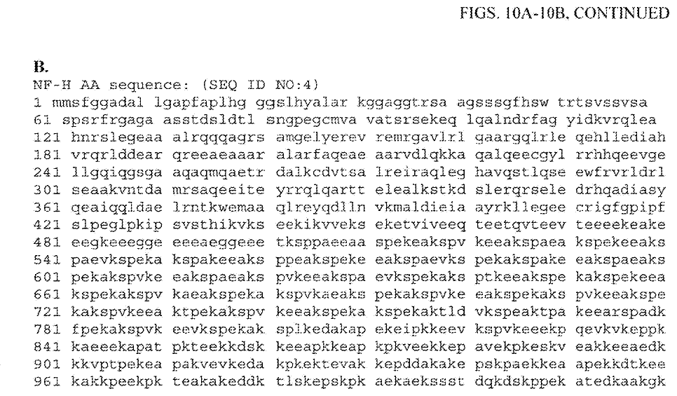

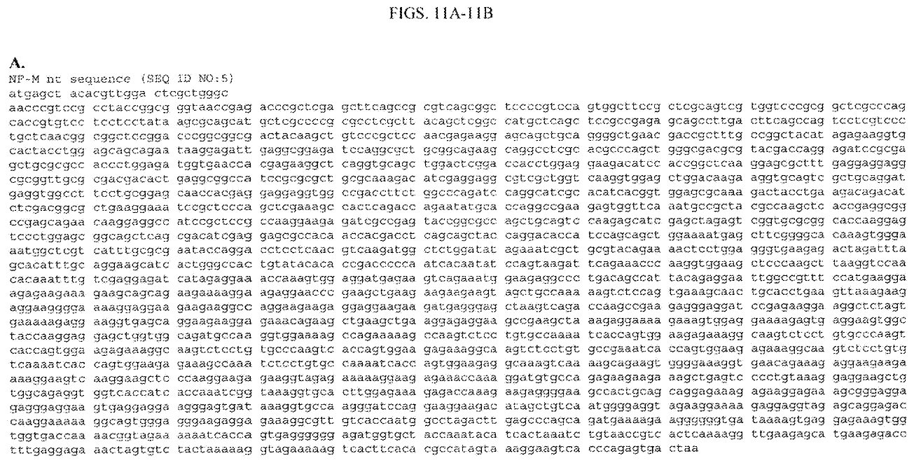

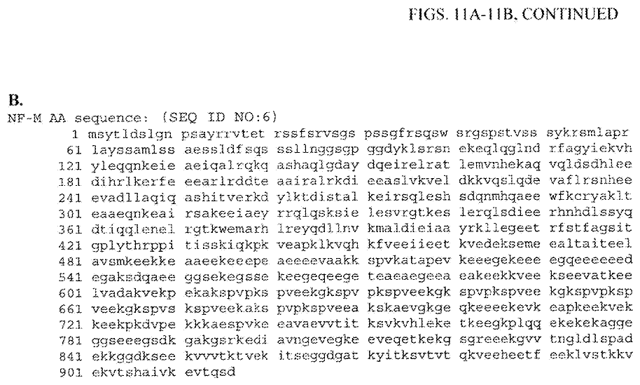

Skin fibroblasts were collected from 3 sporadic ALS patients who do not have familial history of ALS. Induced PSC lines were obtained from the ALS patients' fibroblasts using non-integrating Sendai virus. Differentiation of the ALS lines indicated that MNs and non-MNs can be generated efficiently. Immunohistochemical staining for NF-H, NF-M, and NF-L showed NF aggregations in MNs but not in non-MNs (FIG. 8), highlighting the recapitulation of disease hallmarks by the iPSC model. RT-qPCR analysis indicated that NF-L mRNA, but not NF-H mRNA or NF-M mRNA, was down-regulated in MNs but not in non-MNs that were differentiated from sporadic ALS iPSCs for 31 days (day−10 after plating day−21 cells) (FIG. 8, MN data are shown). Such a striking similarity to the phenotypes observed in SOD1 familial ALS MNs highlights a common mechanism underlying MN degeneration.

### Example 6—NF Aggregates Observed in MNs from Sporadic ALS Subjects and ALS Subjects Having a TDP43 Mutation

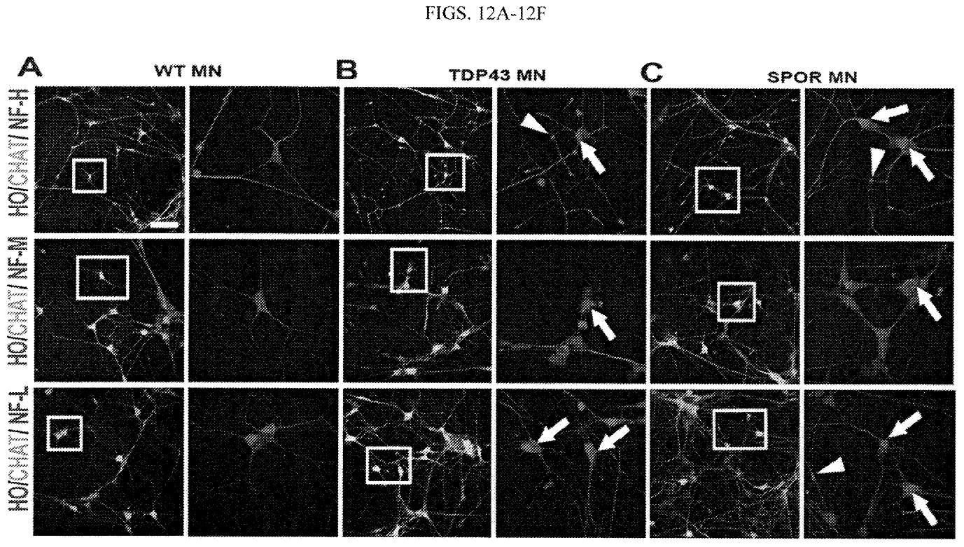

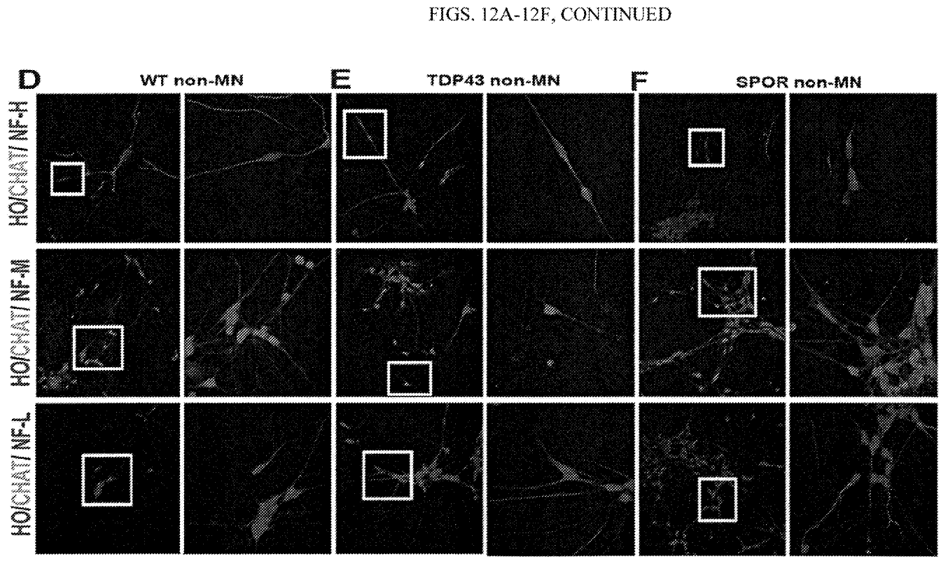

To determine if NF aggregation occurs in ALS patients with other mutations or those without known genetic defects, we established iPSCs from a patient with TDP43 (TAR DNA-binding protein 43) mutation (G298S) and two sporadic ALS patients (no known genetic defects) Sendai virus. As seen with SOD1 mutant ALS cells, iPSCs from TDP43 or sporadic ALS patients differentiated to motor neurons with similar efficiency. Interestingly, immunostaining for NF-H, NF-M and NF-L indicated that the both TDP43 and sporadic ALS MNs contained distinct, inclusion-like focal accumulation of immunoreactive products (FIG. 12). The aggregates were present both in cell body and neurites. The number of NF inclusions in both cell body and on neurites increased over time. The rates of neurofilament aggregation were significantly higher in ALS MNs compared to that in non-ALS MNs (P<0.05) (FIG. 12). The average size of each NF inclusion was approximately 2.94-3.72×10⁻⁴ mm² in cell bodies and approximately 3.75-5.53×10⁻⁶ mm² in neurites at Day 10.

Together, these data demonstrate that NF aggregation is a common motor neuron pathology to all ALS patients (those having genetic defects and those without known genetic defects). These data also suggest there is a common underlying mechanism for ALS pathogenesis.

1. A method for protecting against axonal or neurite degeneration in a subject, the method comprising:
administering a nucleic acid sequence encoding NF-L to a tissue of the subject comprising a neuron,
wherein the nucleic acid sequences are targeted to the neurons of the tissue, and
wherein expression of NF-L protects the targeted neurons from axonal or neurite degeneration.

2. The method of claim 1, wherein the subject is suffering from having amyotrophic lateral sclerosis (ALS) or Spinal Muscle Atrophy (SMA).

3. The method of claim 1, wherein the nucleic acid sequences are administered in a vector.

4. The method of claim 3, wherein vector is a virus or virus-derived vector.

5. The method of claim 4, wherein the virus is an adenovirus, retrovirus, herpes virus, or adeno-associated virus.

6. A recombinant nucleic acid molecule comprising a motor neuron-specific promoter operably linked to a nucleic acid sequence encoding a human NF-L polypeptide.

7. A vector comprising the recombinant nucleic acid molecule of claim 6.

8. The vector of claim 7, wherein vector is a plasmid.

9. The vector of claim 7, wherein vector is a virus or virus-derived vector.

10. The vector of claim 9, wherein the virus is selected from the group consisting of an adenovirus, retrovirus, herpes virus, and adeno-associated virus.

11. The vector of claim 7, wherein the vector is replication defective adenovirus.

12. A kit for diagnosing a subject for a neurodegenerative disease, the kit comprising: an oligonucleotide primer capable of hybridizing to a portion of a NF-L target nucleic acid, a reference corresponding to a level of NF-L target nucleic acid, a buffer or reagent, and a container.

13. The kit of claim 12, wherein the oligonucleotide primer comprises the nucleotide sequence of SEQ ID NO:7 or 8.

14. The kit of claim 12, wherein the neurodegenerative disease is amyotrophic lateral sclerosis (ALS), Charcot-Marie-Tooth disease (CMT), or Spinal Muscle Atrophy (SMA).

15. A method for protecting against axonal or neurite degeneration in a subject, the method comprising:
administering an effective amount of NF-L polypeptide or NF-L mRNA to a tissue of the subject that comprises a neuron,
wherein the NF-L polypeptide or NF-L mRNA are targeted to the neurons of the tissue, and
wherein expression of NF-L protects the targeted neurons from axonal or neurite degeneration.

16. The method of claim 15, wherein the subject is suffering from having amyotrophic lateral sclerosis (ALS) or Spinal Muscle Atrophy (SMA).

17. The method of claim 15, wherein the NF-L polypeptide or NF-L mRNA is administered systemically or via continuous or targeted production of a target gene product in vivo.


SEQUENCE LISTING (8 sequences)

SEQ ID NO: 1 - DNA, 1632 bp, Homo sapiens
  source 1..1632: mol_type = genomic DNA
atgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattga

SEQ ID NO: 2 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 3 - DNA, 3063 bp, Homo sapiens
  source 1..3063: mol_type = genomic DNA
atgatgagcttcggcggcgcggacgcgctgctgggcgccccgttcgcgccgctgcatggcggcggcagcctccactacgcgctagcccgaaagggtggcgcaggcgggacgcgctccgccgctggctcctccagcggcttccactcgtggacacggacgtccgtgagctccgtgtccgcctcgcccagccgcttccgtggcgcaggcgccgcctcaagcaccgactcgctggacacgctgagcaacgggccggagggctgcatggtggcggtggccacctcacgcagtgagaaggagcagctgcaggcgctgaacgaccgcttcgccgggtacatcgacaaggtgcggcagctggaggcgcacaaccgcagcctggagggcgaggctgcggcgctgcggcagcagcaggcgggccgctccgctatgggcgagctgtacgagcgcgaggtccgcgagatgcgcggcgcggtgctgcgcctgggcgcggcgcgcggtcagctacgcctggagcaggagcacctgctcgaggacatcgcgcacgtgcgccagcgcctagacgacgaggcccggcagcgagaggaggccgaggcggcggcccgcgcgctggcgcgcttcgcgcaggaggccgaggcggcgcgcgtggacctgcagaagaaggcgcaggcgctgcaggaggagtgcggctacctgcggcgccaccaccaggaagaggtgggcgagctgctcggccagatccagggctccggcgccgcgcaggcgcagatgcaggccgagacgcgcgacgccctgaagtgcgacgtgacgtcggcgctgcgcgagattcgcgcgcagcttgaaggccacgcggtgcagagcacgctgcagtccgaggagtggttccgagtgaggctggaccgactgtcggaggcagccaaggtgaacacagacgctatgcgctcagcgcaggaggagataactgagtaccggcgtcagctgcaggccaggaccacagagctggaggcactgaaaagcaccaaggactcactggagaggcagcgctctgagctggaggaccgtcatcaggccgacattgcctcctaccaggaagccattcagcagctggacgctgagctgaggaacaccaagtgggagatggccgcccagctgcgagaataccaggacctgctcaatgtcaagatggctctggatatagagatagccgcttacagaaaactcctggaaggtgaagagtgtcggattggctttggcccaattcctttctcgcttccagaaggactccccaaaattccctctgtgtccactcacataaaggtgaaaagcgaagagaagatcaaagtggtggagaagtctgagaaagaaactgtgattgtggaggaacagacagaggagacccaagtgactgaagaagtgactgaagaagaggagaaagaggccaaagaggaggagggcaaggaggaagaagggggtgaagaagaggaggcagaagggggagaagaagaaacaaagtctcccccagcagaagaggctgcatccccagagaaggaagccaagtcaccagtaaaggaagaggcaaagtcaccggctgaggccaagtccccagagaaggaggaagcaaaatccccagccgaagtcaagtcccctgagaaggccaagtctccagcaaaggaagaggcaaagtcaccgcctgaggccaagtccccagagaaggaggaagcaaaatctccagctgaggtcaagtcccccgagaaggccaagtccccagcaaaggaagaggcaaagtcaccggctgaggccaagtctccagagaaggccaagtccccagtgaaggaagaagcaaagtcaccggctgaggccaagtccccagtgaaggaagaagcaaaatctccagctgaggtcaagtccccggaaaaggccaagtctccaacgaaggaggaagcaaagtcccctgagaaggccaagtccccagagaaggaagaggccaagtcccctgagaaggccaagtccccagtgaaggcagaagcaaagtcccctgagaaggccaagtccccagtgaaggcagaagcaaagtcccctgagaaggccaagtccccagtgaaggaagaagcaaagtcccctgagaaggccaagtccccagtgaaggaagaagcaaagtcccctgagaaggccaagtccccagtgaaggaagaagcaaagacccccgagaaggccaagtccccagtgaaggaagaagctaagtccccagagaaggccaagtccccagagaaggccaagactcttgatgtgaagtctccagaagccaagactccagcgaaggaggaagcaaggtcccctgcagacaaattccctgaaaaggccaaaagccctgtcaaggaggaggtcaagtccccagagaaggcgaaatctcccctgaaggaggatgccaaggcccctgagaaggagatcccaaaaaaggaagaggtgaagtccccagtgaaggaggaggagaagccccaggaggtgaaagtcaaagagcccccaaagaaggcagaggaagagaaagcccctgccacaccaaaaacagaggagaagaaggacagcaagaaagaggaggcacccaagaaggaggctccaaagcccaaggtggaggagaagaaggaacctgctgtcgaaaagcccaaagaatccaaagttgaagccaagaaggaagaggctgaagataagaaaaaagtccccaccccagagaaggaggctcctgccaaggtggaggtgaaggaagacgctaaacccaaagaaaagacagaggtagccaagaaggaaccagatgatgccaaggccaaggaacccagcaaaccagcagagaagaaggaggcagcaccggagaaaaaagacaccaaggaggagaaggccaagaagcctgaggagaaacccaagacagaggccaaagccaaggaagatgacaagaccctctcaaaagagcctagcaagcctaaggcagaaaaggctgaaaaatcctccagcacagaccaaaaagacagcaagcctccagagaaggccacagaagacaaggccgccaaggggaagtaa

SEQ ID NO: 4 - protein, 1020 aa, Homo sapiens
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 5 - DNA, 2751 bp, Homo sapiens
  source 1..2751: mol_type = genomic DNA
atgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccgcgcctcgcttacagctcggccatgctcagctccgccgagagcagccttgacttcagccagtcctcgtccctgctcaacggcggctccggacccggcggcgactacaagctgtcccgctccaacgagaaggagcagctgcaggggctgaacgaccgctttgccggctacatagagaaggtgcactacctggagcagcagaataaggagattgaggcggagatccaggcgctgcggcagaagcaggcctcgcacgcccagctgggcgacgcgtacgaccaggagatccgcgagctgcgcgccaccctggagatggtgaaccacgagaaggctcaggtgcagctggactcggaccacctggaggaagacatccaccggctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccggcgccagctgcagtccaagagcatcgagctagagtcggtgcgcggcaccaaggagtccctggagcggcagctcagcgacatcgaggagcgccacaaccacgacctcagcagctaccaggacaccatccagcagctggaaaatgagcttcggggcacaaagtgggaaatggctcgtcatttgcgcgaataccaggacctcctcaacgtcaagatggctctggatatagaaatcgctgcgtacagaaaactcctggagggtgaagagactagatttagcacatttgcaggaagcatcactgggccactgtatacacaccgacccccaatcacaatatccagtaagattcagaaacccaaggtggaagctcccaagcttaaggtccaacacaaatttgtcgaggagatcatagaggaaaccaaagtggaggatgagaagtcagaaatggaagaggccctgacagccattacagaggaattggccgtttccatgaaggaagagaagaaagaagcagcagaagaaaaggaagaggaacccgaagctgaagaagaagaagtagctgccaaaaagtctccagtgaaagcaactgcacctgaagttaaagaagaggaaggggaaaaggaggaagaagaaggccaggaagaagaggaggaagaagatgagggagctaagtcagaccaagccgaagagggaggatccgagaaggaaggctctagtgaaaaagaggaaggtgagcaggaagaaggagaaacagaagctgaagctgaaggagaggaagccgaagctaaagaggaaaagaaagtggaggaaaagagtgaggaagtggctaccaaggaggagctggtggcagatgccaaggtggaaaagccagaaaaagccaagtctcctgtgccaaaatcaccagtggaagagaaaggcaagtctcctgtgcccaagtcaccagtggaagagaaaggcaagtctcctgtgcccaagtcaccagtggaagagaaaggcaagtctcctgtgccgaaatcaccagtggaagagaaaggcaagtctcctgtgtcaaaatcaccagtggaagagaaagccaaatctcctgtgccaaaatcaccagtggaagaggcaaagtcaaaagcagaagtggggaaaggtgaacagaaagaggaagaagaaaaggaagtcaaggaagctcccaaggaagagaaggtagagaaaaaggaagagaaaccaaaggatgtgccagagaagaagaaagctgagtcccctgtaaaggaggaagctgtggcagaggtggtcaccatcaccaaatcggtaaaggtgcacttggagaaagagaccaaagaagaggggaagccactgcagcaggagaaagagaaggagaaagcgggaggagagggaggaagtgaggaggaagggagtgataaaggtgccaagggatccaggaaggaagacatagctgtcaatggggaggtagaaggaaaagaggaggtagagcaggagaccaaggaaaaaggcagtgggagggaagaggagaaaggcgttgtcaccaatggcctagacttgagcccagcagatgaaaagaaggggggtgataaaagtgaggagaaagtggtggtgaccaaaacggtagaaaaaatcaccagtgaggggggagatggtgctaccaaatacatcactaaatctgtaaccgtcactcaaaaggttgaagagcatgaagagacctttgaggagaaactagtgtctactaaaaaggtagaaaaagtcacttcacacgccatagtaaaggaagtcacccagagtgactaa

SEQ ID NO: 6 - protein, 916 aa, Homo sapiens
MSYTLDSLGNPSAYRRVTETRSSFSRVSGSPSSGFRSQSWSRGSPSTVSSSYKRSMLAPRLAYSSAMLSSAESSLDFSQSSSLLNGGSGPGGDYKLSRSNEKEQLQGLNDRFAGYIEKVHYLEQQNKEIEAEIQALRQKQASHAQLGDAYDQEIRELRATLEMVNHEKAQVQLDSDHLEEDIHRLKERFEEEARLRDDTEAAIRALRKDIEEASLVKVELDKKVQSLQDEVAFLRSNHEEEVADLLAQIQASHITVERKDYLKTDISTALKEIRSQLESHSDQNMHQAEEWFKCRYAKLTEAAEQNKEAIRSAKEEIAEYRRQLQSKSIELESVRGTKESLERQLSDIEERHNHDLSSYQDTIQQLENELRGTKWEMARHLREYQDLLNVKMALDIEIAAYRKLLEGEETRFSTFAGSITGPLYTHRPPITISSKIQKPKVEAPKLKVQHKFVEEIIEETKVEDEKSEMEEALTAITEELAVSMKEEKKEAAEEKEEEPEAEEEEVAAKKSPVKATAPEVKEEEGEKEEEEGQEEEEEEDEGAKSDQAEEGGSEKEGSSEKEEGEQEEGETEAEAEGEEAEAKEEKKVEEKSEEVATKEELVADAKVEKPEKAKSPVPKSPVEEKGKSPVPKSPVEEKGKSPVPKSPVEEKGKSPVPKSPVEEKGKSPVSKSPVEEKAKSPVPKSPVEEAKSKAEVGKGEQKEEEEKEVKEAPKEEKVEKKEEKPKDVPEKKKAESPVKEEAVAEVVTITKSVKVHLEKETKEEGKPLQQEKEKEKAGGEGGSEEEGSDKGAKGSRKEDIAVNGEVEGKEEVEQETKEKGSGREEEKGVVTNGLDLSPADEKKGGDKSEEKVVVTKTVEKITSEGGDGATKYITKSVTVTQKVEEHEETFEEKLVSTKKVEKVTSHAIVKEVTQSD

SEQ ID NO: 7 - DNA, 22 bp, synthetic construct
  misc_feature 1..22: note = oligonucleotide primer
  source 1..22: mol_type = other DNA
atgagttccttcagctacgagc

SEQ ID NO: 8 - DNA, 20 bp, synthetic construct
  misc_feature 1..20: note = oligonucleotide primer
  source 1..20: mol_type = other DNA
ctgggcatcaacgatccaga